(19) United States
(12) Patent Application Publication
(10) Pub. No.: US 2020/0411185 A1
(43) Pub. Date: Dec. 31, 2020

(54) ADAPTIVE INTERVENTIONS FOR GASTROINTESTINAL HEALTH CONDITIONS

(71) Applicant: Mahana Therapeutics, Inc., San Francisco, CA (US)

(72) Inventors: Megan Leigh Oser, San Francisco, CA (US); Robert Bradley Paull, Tiburon, CA (US)

(21) Appl. No.: 16/906,085

(22) Filed: Jun. 19, 2020

Related U.S. Application Data
(60) Provisional application No. 62/867,275, filed on Jun. 27, 2019.

(51) Int. Cl. G16H 50/20; G16H 50/30; G16H 10/60; G16H 20/70; G16H 20/10; G16H 80/00; G16H 40/67; A61B 5/00

(52) U.S. Cl. CPC G16H 50/20; G16H 50/30; G16H 10/60; A61B 5/4216; G16H 20/10; G16H 80/00; G16H 40/67; G16H 20/70

(57) ABSTRACT

A method and system for GI health condition monitoring and improvement, where the method includes steps for receiving signals associated with the GI condition, the signals encoding physiological data, behavioral data, environmental stress data, emotional data, and cognitive data of the user; determining a characterization of the GI condition upon processing the set of signals with a model; based upon the characterization, modulating content of a treatment comprising a set of components, the set of components comprising a subset of cognitive behavioral therapy (CBT) components for improving a state of the user; and administering the treatment to the user. The system and method can be provided as a prescription digital therapeutic for improving patient outcomes for users with GI health conditions.

### CROSS-REFERENCE TO RELATED APPLICATIONS

This application claims the benefit of U.S. Provisional Application No. 62/867,275 filed on 27 Jun. 2019, which is incorporated in its entirety herein by this reference.

### TECHNICAL FIELD

This invention relates generally to the fields of gastrointestinal health and digital therapeutics, and more specifically to a new and useful system and method for providing adaptive interventions for gastrointestinal health conditions in the fields of gastrointestinal health and digital therapeutics.

### BACKGROUND

Gastrointestinal (GI) health conditions and other digestive disorders have significant impacts worldwide. An estimated 60-70 million people in the U.S. alone have diagnosed GI health conditions, with millions more undiagnosed individuals experiencing symptoms but failing to receive treatment. In relation to treatment and therapy for GI health conditions, current approaches focus on reducing or eliminating physiological symptoms, by implementation of medication regimens, supplement regimens, diet changes, and/or lifestyle changes. However, GI health conditions have other adverse effects on lives of patients due to the nature of symptoms, and current approaches to treatment fail to address such adverse effects. Furthermore, current methods of improving patient states associated with GI health are limited in relation to: educating patients regarding standard and non-standard treatment options; detecting, in real or near-real time, states of symptom severity in non-invasive manners; and delivering therapy in a customized, and adaptive manner.

Thus, there is a need in the fields of GI health and digital therapeutics to create a new and useful system and method for detecting patient states and providing adaptive interventions for improving patient states.

### BRIEF DESCRIPTION OF THE FIGURES

FIG. 1 depicts a schematic of a system for providing adaptive interventions for gastrointestinal health conditions, according to one or more embodiments.

FIG. 2A depicts a flowchart of a method for providing adaptive interventions for gastrointestinal health conditions, according to one or more embodiments.

FIG. 2B depicts a flowchart of a method for providing adaptive interventions for gastrointestinal health conditions, according to one or more embodiments.

FIGS. 3A and 3B depict flows and examples of embodiments of determining severity of a gastrointestinal health condition.

FIG. 4 depicts a flowchart of an example pre-assessment and onboarding process of a method providing adaptive interventions for gastrointestinal health conditions.

FIG. 5 depicts an example of formation of a personalized gastrointestinal health condition model for a user.

FIG. 6 depicts examples of application aspects of a program for personalized gastrointestinal health condition monitoring and improvement.

FIG. 7 depicts a schematic of architecture implemented for delivery of intervention regimen components.

FIGS. 8A-8E depict example schematics of conditional branching architecture implemented for delivery of intervention regimen components.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Benefits

The inventions covered by the system and method can confer several benefits over conventional systems and methods, and such inventions are further implemented into many practical applications related to improvement of user health.

The invention(s) can employ non-traditional systems and methods for providing interventions to patients exhibiting symptoms associated with one or more GI health conditions. In particular, the invention(s) can deliver psychological-based interventions, such as cognitive behavioral therapy (CBT)-based interventions and other interventions (described in more detail below) to users, by way of a platform having components implemented in a mobile device environment and/or other computer or internet-based architecture. The invention(s) thus use components of the platform to process user data, deliver interventions, and monitor user interactions with such interventions in a manner that cannot be practically implemented by the human mind.

The invention(s) can also provide interventions that are tailored to individual users suffering from symptoms (e.g., associated with digestion, defecation, other stooling symptoms, pain, social/interpersonal effects, emotional effects, cognitive effects, behavioral effects, etc.), in a customized manner, with implementation of real-time or near real-time assessments of data from multiple sources (e.g., electronic health record sources, self-report sources, sensor sources, etc.).

The invention(s) can also be used for acquisition of data (e.g., health data, biometric data, user demographic data, user behavior data, etc.) from multiple data sources. In this regard, the invention(s) can also be used for generation of training datasets, whereby the training datasets can be used for training machine learning models (e.g., neural networks, etc.) that take input data pertaining to patients and produce outputs that can be used to guide customization of interventions.

The invention(s) can also be used to provide automated delivery of health-promoting or improving interventions, automated tracking/monitoring of user interactions with such interventions, automated communications with users (e.g., through transmission of notifications), and/or automated delivery of modified interventions to users, through a mobile device application platform and/or other platform (e.g., web platform). Such interventions can also be delivered as digital therapeutics (e.g., alone as a monotherapy or in combination with other therapeutics, such as medications and/or medical devices), software intended to diagnose and/or treat and/or improve symptoms or health-related quality of life, in collaboration with healthcare providers, health insurers, and/or other entities in the healthcare system. The invention(s) can also employ non-traditional systems and methods for delivering prescription digital therapeutics (PDT) for improving patient health (e.g., in relation to disease management), whereby digital therapeutics are prescribed through healthcare providers (e.g., with associated billing codes).

Additionally or alternatively, the invention(s) can include systems and methods for improving patient states (e.g., in the context of health, symptoms, disease progression, quality of life, and other contexts).

Additionally or alternatively, the system and/or method can confer any other suitable benefit.

### 2. System

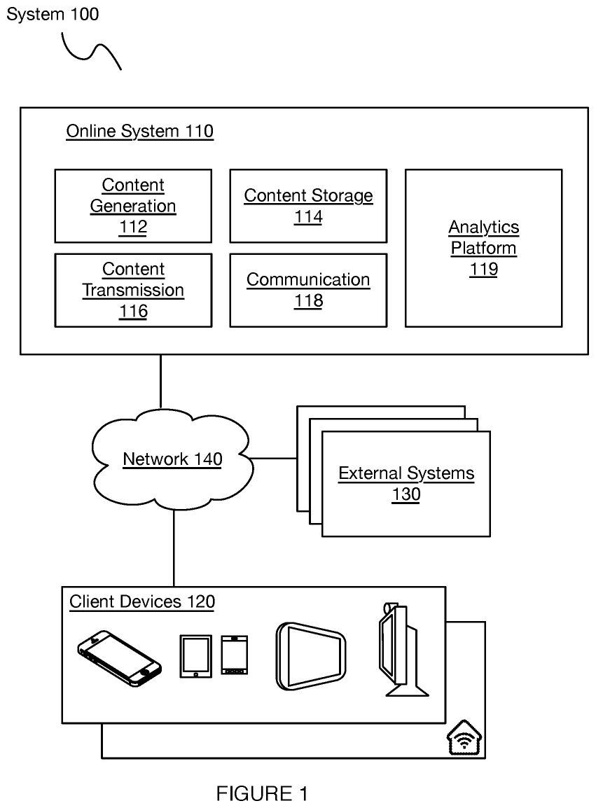

As shown in FIG. 1, an embodiment of a system 100 for providing adaptive interventions for gastrointestinal (GI) health conditions includes: an online system 110 for digital content associated with the adaptive interventions, one or more client devices including client device 120 for delivering the adaptive interventions to one or more users, one or more external systems including external system 130, and a network 140 for data transmission between the online system 110, the client device(s) 120, and the external system(s) 130. The system 100 includes functionality for educating subjects (e.g., patients, users of the platform, etc.) regarding treatment and therapy options in the context of improving symptoms associated with GI health; detecting, in real or near-real time, states of GI health condition symptom severity in non-invasive manners; and delivering interventions in a customized, and adaptive manner to one or more users exhibiting GI health condition symptoms. In some embodiments, the system 100 can provide tailored cognitive behavioral therapies (CBTs) or other therapeutic pathways, such as Acceptance Commitment Therapy (ACT), Gut-Directed Hypnotherapy, or Mindfulness-Based Cognitive Therapy (MCBT), for patients in an adaptive and customizable manner. While GI health condition symptoms are indicated, variations of the system 100 can be adapted for generation and provision of interventions for systems associated with other health conditions.

### 2.1 System—Online System

The online system 110 functions to generate, store, and transmit digital content associated with the adaptive interventions, according to algorithms that allow the online system 110 to deliver (or guide delivery) of interventions to subjects in a timely and customized manner. The online system no thus procures and allows subjects of the system 100 to access digital content associated with one or more health interventions in an active or passive manner, in order to improve the subject(s)' ability to manage GI health condition symptoms or other symptoms. The online system no can include content generation components 112, content storage components 114, content transmission components 116, communication elements 118, and/or analytic platform 119 elements implemented in computer architecture. The online system no can additionally or alternatively include any other suitable subsystems or components associated with provision of adaptive interventions and/or monitoring of subject health condition states.

In relation to content generation components 112, the online system no can include computing architecture configured for generation of interactive digital objects in computer-readable formats, where such interactive digital objects can be included in therapeutic interventions (e.g., modules of an application or program) provided to subjects exhibiting one or more GI health condition symptoms. In variations, the content generation components 112 can include architecture for generation of content in one or more of: visual formats (e.g., with image objects, video objects, etc.), audible formats, haptic formats, and any other suitable formats. Such content can be delivered through output devices of other components of the system 100, such as display components (e.g., of a device, of an augmented reality device, of a virtual reality device, etc.), speaker components, haptic output device components, and/or any other suitable components.

In relation to content storage components 114, the online system 110 can include architecture for storage and retrieval of computer-readable media associated with digital content and/or other objects. Data storage systems can be associated with any suitable format, and include components configured for cloud and/or non-based cloud computing. In particular embodiments, the information stored in the content storage components 114 can be organized according to specific data structures (e.g., with relational, columnar, correlation, or other suitable architecture). Stored content can be associated with various digital objects (e.g., graphical/textual/audio/visual/haptic objects associated with content, and/or rearrangement of objects within particular environments, as associated with therapeutics and/or communications between entities, as described in more detail below).

In relation to content transmission components 116, the online system 110 can be configured to transmit content over wired and/or wireless interfaces, through network 140 (described in more detail below). As such, the content transmission components 116 of the online system 110 can include interfaces to the network 140, for content transmission to client devices and/or external systems.

In relation to communication elements 118, the online system 110 can include elements that enable communications between subjects and other entities (e.g., care providers, coaches associated with health interventions, other subjects, etc.) in text format, in audio format, and/or in any other suitable formats. In examples, the online system 110 can support messaging, calling, and/or any other suitable communication types using web or other application-based communication subsystems.

In relation to analytics, the online system 110 can include architecture for an analytics platform 119 for performing analytics in relation to generation of interventions (e.g., digital therapeutics as monotherapies, digital therapeutics as combinatorial therapies), evaluation of performance of interventions (e.g., in relation to performance, in relation to effectiveness, etc.), modification of interventions (e.g., in relation to content aspects, in relation to frequency aspects, etc.), provision of interventions (e.g., delivery method, etc.), generating and processing training data for refinement of models for intervention generation and provision (as described in relation to the processes of Section 3 below), and other architecture for performing analytics.

One or more portions of the online system 10 can include processing subsystem components comprising non-transitory media storing instructions for executing one or more method steps described below. The processing subsystem components can be distributed across the online system 110, client devices 120, and external systems 130, or organized in another suitable manner.

The online system no can be implemented in a network-addressable computing system that can host one or more components for generating, storing, receiving, and sending data (e.g., content-related data, user-related data, data related to entities associated with various therapeutics, etc.). The online system 110 can thus be accessed by the other components of the system 100 either directly or via network 140 described below. In particular embodiments, the online system no can include one or more servers (e.g., unitary servers, distributed servers spanning multiple computers or multiple datacenters, etc.). The servers can include one or more server types (e.g., web server, messaging servers, advertising servers, file servers, application servers, exchange servers, database servers, proxy servers, etc.) for performing functions or processes described. In particular embodiments, each server can thus include one or more of: hardware, software, and embedded logic components for carrying out the appropriate functionalities associated with the method(s) described in Section 3 below.

### 2.2 System—Client Devices and External Systems

The client device(s) 120 function to deliver the adaptive interventions generated and/or stored by the online system no to subjects exhibiting GI health condition systems in a timely manner. The client device(s) 120 can include computing components, input devices, and/or output devices providing interfaces for receiving subject inputs and transmitting digital content data and/or sensor-derived data over the network 140 (described in more detail below). In embodiments, the client device(s) 120 can include one or more of: mobile computing devices (e.g., a smartphone a personal digital assistant); a conventional computing system (e.g., desktop computer, laptop computer); a tablet computing device; a wearable computing device (e.g., a wrist-borne wearable computing device, a head-mounted wearable computing device, an apparel-coupled wearable computing device); a toilet-interfacing computing device; and any other suitable computing device.

In variations, the client device(s) 120 can be configured to store and/or execute an application (e.g., mobile application, web application) that allows a user of the client device 120 to interact with the online system 110 by way of the network 140, in order to receive digital content associated with one or more interventions and/or provide data associated with survey responses, sensor-derived data associated with interactions with such interventions, and/or any other suitable data. In relation to providing treatments, the client device(s) 120 can include operation modes for administering treatments to the user (e.g., in relation to providing prescription digital therapeutics upon diagnosis of the GI condition of the user, in relation to providing medications, in relation to providing pain management therapies, etc.).

The external system(s) 130 function to transmit data (e.g., 3^{rd} party data) and/or receive data (e.g., 3^{rd} party data) associated with interventions and/or user data (e.g., patient data). The external system(s) 130 can include systems associated with electronic health records (EHRs) of the subject(s), systems associated with collection and/or storage of subject data (e.g., biometric data, behavioral data, social network data, communication data, etc.), systems associated with care providers (e.g., health insurance providers, health care practitioners, etc.), and/or any other suitable systems. In embodiments, the external system(s) can provide applications for communicating data in a manner that is protective of personal health information (PHI) and/or other sensitive subject data. Additionally or alternatively, the external system(s) can be associated with 3^{rd} party content generators and generate digital content in visual formats, audible formats, haptic formats, and/or any other suitable formats.

The external system(s) 130 and/or client device(s) 120 can be configured to interact with the online system 110 by way of an application programming interface (API) executing on a native operating system of the external system(s) 130 and/or client device(s), in order to access API-associated data associated with the interventions, subject health records, and/or other data (e.g., biometric data, subject behavior data through social networks, communication data through communication subsystems, etc.).

As indicated above, the external system(s) 130 and/or client devices 120 can further include sensing components configured to generate data from which subject biometrics and/or behaviors can be extracted. In relation to biometric data, the external system(s) 130 and/or client devices 120 can include sensing components associated with one or more of: activity of a subject (e.g., through accelerometers, gyroscopes, motion coprocessing devices, etc.); facial expressions of the subject (e.g., through eye tracking, through image/video processing) for determination of cognitive states (e.g., associated with depression, anxiety, emotions, etc.) and/or performance of activities and/or interacting with content provided through the intervention regimen; physiological and/or psychological stress of a subject (e.g., in relation to respiration parameters, in relation to cardiovascular parameters, in relation to galvanic skin response, in relation to neurological activity, in relation to other stress biometrics, etc.); sleep behavior of a subject (e.g., with a sleep-monitoring device); digestive health of a subject (e.g., in relation to microbiome composition, in relation to stool-based assays, in relation to urine-based assays, in relation to smart-pill devices, in relation to smart toilet devices); and any other suitable sensors or devices from which biometric signals can be acquired for assessment of subject health.

In relation to behavioral data, the external system(s) 130 and/or client devices 120 can include components for extracting behavioral data associated with communications and social behavior, which can be indicative of changes in subject health associated with different symptoms. Such components can include location sensors (e.g., direct location sensors, location sensing modules based on connections to local networks, triangulation systems, etc.) for tracking user motility and/or other behavior patterns, components associated with API access to social networking data, components associated with messaging communication behavior (e.g., components for accessing SMS or other messaging application data of a subject, with respect to messaging entities, messaging content, etc.), components associated with calling communication behavior (e.g., in relation to inbound/outbound calls, in relation to call duration, in relation to call content, etc.), data from digital assistants (e.g., voice-activated digital assistants) and any other suitable components from which behavioral data can be extracted.

### 2.3 System—Network

The network 140 functions to enable data transmission between the online system 110, the client device(s) 120, and the external system(s) 130, in relation to detection of subject states of wellbeing (e.g., with respect to GI health condition symptoms). The network 140 can include a combination of one or more of local area networks and wide area networks, and/or can include wired and/or wireless connections to the network 140. The network 140 can implement communication linking technologies including one or more of: Ethernet, worldwide interoperability for microwave access (WiMAX), 802.11 architecture (e.g., Wi-Fi, etc.), 3G architecture, 4G architecture, 5G architecture, long term evolution (LTE) architecture, code division multiple access (CDMA) systems, digital subscriber line (DSL) architecture, and any other suitable technologies for data transmission.

In variations, the network 140 can be configured for implementation of networking protocols and/or formats including one or more of: hypertext transport protocol (HTTP), multiprotocol label switching (MPLS), transmission control protocol/Internet protocol (TCP/IP), file transfer protocol (FTP), simple mail transfer protocol (SMTP), hypertext markup language (HTML), extensive markup language (XML), and any other suitable protocol/format. The network 140 can also be configured for and/or provide, through communication links, encryption protocols for improving security of subject data transmitted over the network 140.

### 2.4 System—Additional Aspects

The system 100 can include or be configured to interface with other system components associated with generation and/or delivery of adaptive interventions. For instance, the system 100 can include or be associated with environmental control devices configured to affect subject states of wellbeing passively or actively, in relation to the intervention types described in more detail in Section 3 below. In some embodiments, such devices can include environmental control devices, including one or more of: lighting control devices, audio output devices, temperature control devices, and any other suitable environmental control devices. The system 100 can coordinate operation of such devices with delivery of adaptive interventions to subjects, such that aspects of the subject's environment can be modulated in coordination with other therapeutic measures to improve subject wellbeing in relation to GI health condition symptoms or other symptoms. For instance, in variations of the method below, the system 100 can include and/or communicate control instructions for devices in the environment of the users, in order to facilitate control of pain volume, in relation to magnitude of pain/intensity of pain (e.g., by focusing the user on real time environmental changes) and/or to cause improvements in lives of users in another suitable manner.

As such, in one variation, the system 100 can include an output device (e.g., component of client device 120, component of external system 130, etc.) that functions as an environmental control device in an environment of the user, where the processing subsystem further includes instructions for adjusting the operation mode in coordination with monitoring a change in symptoms (e.g., pain symptoms) of the user. Modulation of output device operation modes can thereby produce an adjustment in symptoms (e.g., pain volume) associated with the condition of the user. In examples, the environmental control device can modulate one or more of: an audio output, a thermal parameter adjustment, a visually-observed output, a haptic output, and a light output in the environment.

In another variations, the system 100 can include an output device (e.g., component of client device 120, component of external system 130, etc.) that functions as a communication device for transmitting communications between the user and an entity associated with the user, where the processing subsystem further includes instructions for generating a scripted communication for transmission to an entity associated with the user, in coordination with monitoring a change in a physiological symptoms of the user

The system 100 can, however, be configured to interface or include any other suitable system components.

Embodiments, variations, and examples of one or more components of the system 100 described above can implement one or more embodiments, variations, and examples of the method 200, as described in Section 3 below. The system 100 can additionally or alternatively be configured to implement other methods.

### 3. Method

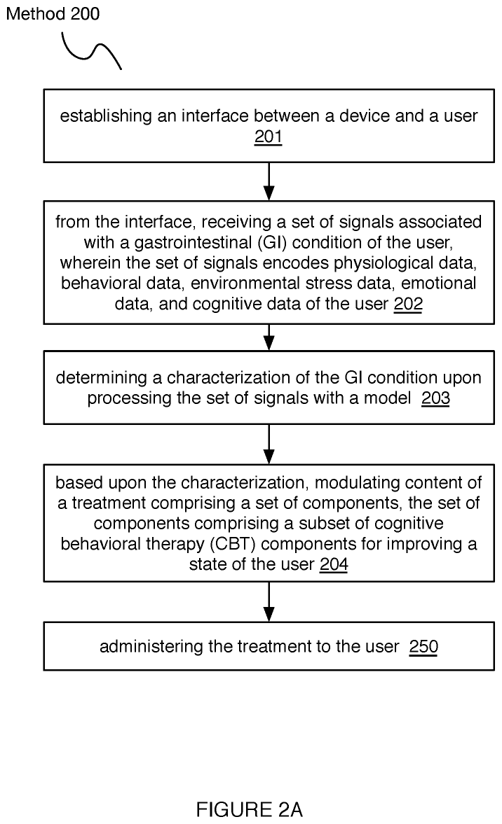

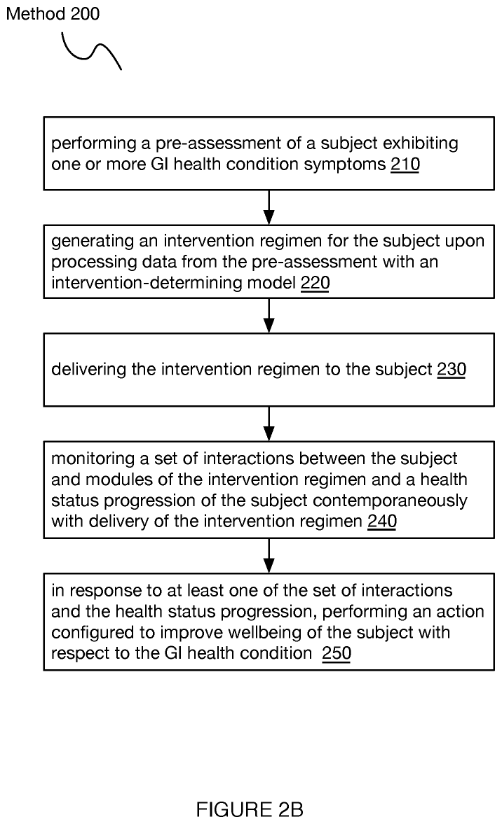

As shown in FIG. 2A, an embodiment of a method 200 for providing adaptive interventions for gastrointestinal (GI) health conditions can include steps for: establishing an interface between a device and a user 201; from the interface, receiving a set of signals associated with a gastrointestinal (GI) condition of the user, wherein the set of signals encodes physiological data, behavioral data, environmental stress data, emotional data, and cognitive data of the user 202; determining a characterization of the GI condition upon processing the set of signals with a model 203; based upon the characterization, modulating content of a treatment comprising a set of components, the set of components comprising a subset of cognitive behavioral therapy (CBT) components for improving a state of the user 204; and administering the treatment to the user 205.

As shown in FIG. 2B, a related embodiment of a method 200 for providing adaptive interventions for gastrointestinal (GI) health conditions can include steps for: performing a pre-assessment of a subject exhibiting one or more GI health condition symptoms 210 (e.g., as associated with steps 201 and 202); generating an intervention regimen for the subject upon processing data from the pre-assessment with a model 220 (e.g., as associated with steps 203 and 204); delivering the intervention regimen to the subject 230 (e.g., as associated with step 205); monitoring a set of interactions between the subject and modules of the intervention regimen and a health status progression of the subject contemporaneously with delivery of the intervention regimen 240; and in response to at least one of the set of interactions and the health status progression, performing an action configured to improve wellbeing and symptoms of the subject with respect to the GI health condition 250. In relation to interactive aspects of the intervention regimen, the method 200 can further include steps for detecting performance of activities associated with the intervention regimen, by the subject; reinforcing user performance or engagement with the intervention regimen; determining undesired levels of performance or engagement with the intervention regimen; and driving improved engagement with the intervention.

The method 200 functions to educate subjects regarding treatment and therapy options in the context of improving symptoms associated with GI health; detect, in real or near-real time, states of GI health condition symptom severity in non-invasive manners; and deliver interventions in a customized, and adaptive manner to one or more users exhibiting GI health condition symptoms. In some embodiments, the method 200 can be used to provide tailored cognitive behavioral therapy (CBT) or other therapeutic pathways to subjects in an adaptive and customizable manner. While GI health condition symptoms are described, variations of the method 200 can be adapted for generation and provision of interventions for systems associated with other health conditions.

Aspects of the method 200, such as provision of components, promotion of interactions with the system by way of an application (e.g., mobile application, web application, etc.), processing of data, performance of analyses (e.g., associated with program efficacy, associated with user symptoms, etc.), model refinement, and other aspects can be performed at desired frequencies (e.g., weekly, more often than weekly, less often than weekly). For instance, in relation to triggered interactions with the system by way of an application (e.g., mobile application, web application, etc.), the method can promote interactions more often than weekly (e.g., daily, 2 times a week, 3 times a week, four times a week, five times a week, six times a week, etc.) or less often than weekly, in relation to reinforcement of skills acquired by the subjects. Furthermore, received data can be processed in real time, or non-real time. However, the method 200 can have delivery and processing aspects associated with other suitable frequencies.

The method 200 can be performed by an embodiment, variation, or example of the system 100 described in Section 2 above (e.g., in relation to processing subsystem components with instructions stored in non-transitory media and other input/output devices); however, the method 200 can additionally or alternatively be performed using any other suitable system components.

### 3.1 Method—Onboarding and Pre-Assessment

In relation to system components described above, an embodiment of the online system, in coordination with the network and a client device, can perform 210 a pre-assessment of a subject exhibiting one or more GI health condition symptoms, contemporaneously with executing an onboarding process with the subject and the online system. Block 210 functions to retrieve data describing characteristics of the subject, preferences of the subject, goals of the subject and/or any other suitable subject features that can be used to provide adaptive interventions in a customized and personalized manner, in order to promote user engagement with the intervention regimen(s) described in subsequent steps of the method 200.

In relation to subjects, Block 210 can include pre-assessing and onboarding subjects and assessing characteristics including one or more of: demographics (e.g., genders, ages, familial statuses, residential location, ethnicities, nationalities, socioeconomic statuses, sexual orientations, etc.), household situations (e.g., living alone, living with family, living with a caregiver, etc.), dietary characteristics (e.g., omnivorous, vegetarian, pescatarian, vegan, reduced carbohydrate consumption, reduced acid consumption, gluten-free, simple carbohydrate, of other dietary restrictions, etc.), levels of activity, levels of alcohol consumption, levels of drug use, psychological symptom severity, levels of mobility (e.g., in relation to distance traveled in a period of time), biomarker statuses (e.g., fecal calprotectin, cholesterol levels, lipid states, blood biomarker statuses, etc.), weight, height, body mass index, genotypic factors, durations of mindfulness (e.g., mindful minutes), and any other suitable characteristic associated with GI health (or other health considerations).

In relation to GI health, the pre-assessment and/or onboarding process performed in Block 210 can identify the subject as having GI health condition symptoms associated with one or more of: irritable bowel syndrome (IBS), inflammatory bowel disease (IBD, such as associated with Crohn's disease or ulcerative colitis), lactose intolerance, gastroesophageal reflux disease (GERD), ulcers (e.g., peptic ulcer disease, gastric ulcers, etc.), hernias, celiac disease, diverticulitis, malabsorption, short bowel syndrome, intestinal ischemia, pancreatitis, cysts, gastritis, esophagitis, achalasia, strictures, anal fissures, hemorrhoids, proctitis, prolapse, gall stones, cholecystitis, cholangitis, GI-associated cancers, bleeding, bloating, constipation, diarrhea, heartburn, incontinence, nausea, vomiting, abdominal pain, swallowing issues, weight maintenance issues, and/or any other suitable symptoms. In relation to the set of signals of Step 201, the set of signals can encode physiological data, behavioral data, environmental stress data, emotional data, and cognitive data of the user, from the pre-assessment, health record access, API access of health monitoring applications, and/or biometric sensors. Furthermore, such signals can be collected repeatedly throughout performance of the methods described.

In more detail with respect to IBS, the pre-assessment can be configured to receive information regarding (or automatically detect, or automatically extract, based upon symptoms, etc.) the subtype(s) of IBS (e.g., IBS-C with predominant constipation, IBS-D with predominant diarrhea, IBS-M with mixed bowel habits) a subject has, in order to prioritize relevant content provided to the subject, in the interests of customizing the program. For instance, if the pre-assessment 210 identifies that the subject is predominantly subtype IBS-C, subsequent portions of the method 200 can prioritize content associated more highly with IBS-C. Subtype identification can, however, be assessed outside of the pre-assessment of Step 210. Furthermore, in relation to subtype identification, prescription digital therapeutics provided by the method 200 and system 100 can be provided as monotherapies, or as complementary therapies. In more detail, complementary therapies for IBS-C can include one or more of: antibiotics, antidepressants, antispasmodics, 5-hydroxytryptamine 4 agonists, over-the-counter laxatives, probiotics, selective C-2 chloride channel activators, and other therapies. In more detail, complementary therapies for IBS-D can include one or more of: antibiotics, antidepressants, anti-diarrheal medications, antispasmodics, 5-hydroxytryptamine 4 agonists, probiotics, and other therapies. In more detail, complementary therapies for IBS-M can include one or more of: antibiotics, antidepressants, antispasmodics, probiotics, and other therapies. Complementary therapies can further include one or more of: psychological treatments, hypnotherapy, acupuncture, herbal therapies, oils, and other therapies.

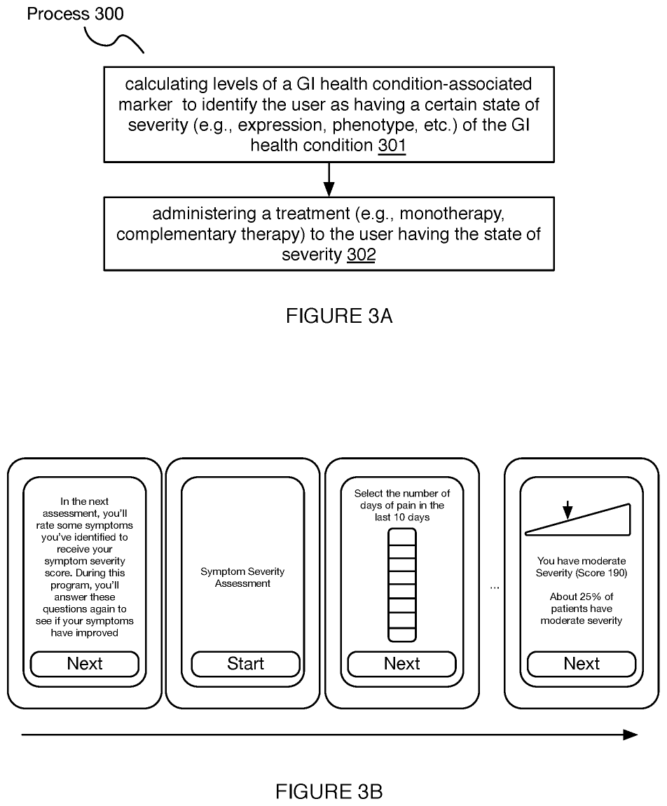

In variations related to monotherapies and complementary therapies, the method 200, as shown in FIG. 3A, can include a process 300 for calculating levels of a GI health condition-associated marker (e.g., from a sample from the user, such as a stool sample or a breath sample, from interactions with the system, etc.) to identify the user as having a certain state of severity (e.g., expression, phenotype, etc.) of the GI health condition 301. In an example, as shown in FIG. 3B, step 301 can be implemented through an application executing at a mobile device or other device associated with the user, where the application prompts inputs from the user pertaining to various symptoms (e.g., pain, defecation, abdominal distension, digestive issues, cognitive symptoms, behavioral effects, etc.) and generates a report indicating severity of the GI health condition (e.g., IBS, IBD, etc.).

The process 300 shown in FIG. 3A can then include administering a treatment (e.g., monotherapy, complementary therapy) to the user having the state of severity 302, where the treatment comprises one or more of the therapies described. Further, in relation to cognitive behavioral therapies vs. other therapies, the method 200 can include adjusting (e.g., decreasing, increasing, maintaining) an amount of a non-CBT treatment provided to the user based upon the state of severity, and/or correspondingly adjusting (e.g., decreasing, increasing, maintaining) an amount of a CBT treatment provided to the user, thereby titrating relative treatment types provided to the user based upon returned outputs of models associated with the methods described. As such, a treatment cocktail can include prescription digital therapeutic aspects and non-prescription digital therapeutic aspects.

In relation to mental health associated with GI health condition symptoms, the pre-assessment and/or onboarding process performed in Block 210 can identify mental health statuses of the subject, in relation to comorbid or non-comorbid conditions (e.g., associated with anxiety, associated with depression, associated with social behavior, etc.), where the intervention regimen described in more detail below can be configured to improve mental health states of the subject in a timely and adaptive manner.

Related data can include psychological and/or disease symptom/clinical profile data that informs selection of high priority CBT components, where examples include one or more of: illness-related ruminations being predominant; symptoms triggered by anticipatory anxiety; aspects adapted for types of reinforcement based on level of anhedonia, as assessed from system-provided tools associated with depression assessment (e.g., upon identification of anhedonia characteristics of the subject, promoting behavioral activation content by the system and response chaining, where response chaining involves linking of effortful avoided tasks to those that are neutral or slightly rewarding); sources of motivation; reward sensitivity (e.g., sensitivity associated with drive and reward responsiveness (e.g., using a BIS/BAS assessment tool); and threat sensitivity. These types of reward processing can then inform a user's responsivity to progress and failure in goal-pursuit. As such, the method 200 can include receiving a reward sensitivity dataset characterizing motivation and reinforcement behavior of the user, and modulating aspects of the treatment upon processing the reward sensitivity dataset with one or more models described. Mental health, reward tendencies and sensitivity, and motivational aspect identification can, however, be assessed outside of the pre-assessment of Step 210.

In relation to user preferences (e.g., with respect to receiving transmissions associated with the intervention regimen), the pre-assessment and/or onboarding process performed in Block 210 can identify user preferences associated with scheduling of content delivery (e.g., in relation to frequencies of content delivery described above) associated with one or more aspects of the intervention regimen, preferred formats (e.g., visual formats, audio formats, haptic formats, etc.) of content delivery, frequency of content delivery, location of user when content is delivered, specific device(s) to which content is delivered, and/or any other suitable user preferences.

In relation to assessing goals of the subject, the pre-assessment and/or onboarding process performed in Block 210 can identify user goals for improving health, in relation to the intervention regimen. Such goals can include one or more of: reduction of anxiety, reduction of negative emotions, reduction of depression symptoms, improvement of sleep behavior, improvement in socialization, improvement of GI health condition symptoms, improvement of medication adherence, improvement in GI-related quality of life, improvement of other health condition symptoms, and/or any other suitable goals. Goals can be organized at a high level of abstraction (e.g., improve sleep behavior), and/or at lower levels of abstraction (e.g., improve quality of sleep, reduce number of symptom-induced disturbances to sleep, etc.).

In relation to performing the pre-assessment and/or onboarding process, the online system and/or other system components can implement surveying tools (e.g., for self-report of data from the subject) and/or non-survey-based tools for acquisition of data. Survey tools can be delivered through an application (e.g., mobile application, web application, etc.) executing on the client device of the subject and/or through another suitable method, where the survey tools can implement architecture for assessing the subject in relation to mental health, pain, GI health symptom severity or disease activity (e.g. IBS-symptom severity scale), types of GI health condition symptoms, and/or other statuses. In examples the surveying tools can be derived from one or more of: a patient health questionnaire (e.g., PHQ-9), an anxiety disorder questionnaire (e.g., GAD-7, PC-PTSD, SCARED), a work and social adjustment scale (WSAS)-derived tool, a pain assessment questionnaire (e.g., numerical rating scale, Wong-Baker faces scale, FLACC scale, CRIES scale, COMFORT scale, McGill scale, Color Analog scale, etc.), a clinical disease activity measurement (e.g., CDAI, PUCAI, Mayo Score) and any other tool or instrument. Survey components can be implemented during pre-assessment of a subject and/or within modules of the intervention regimen, as described in more detail below. As such, the system can include architecture for receiving data derived from the subject (e.g., through sensor components, through survey components, associated with pain characteristics, digestive characteristics, defecation characteristics, and other characteristics), processing the data with one or more models, and returning scores (e.g., measures of symptom severity, etc.). Scores can also be used for tagging user data with symptom severity, in relation to model aspects and model training/refinement described below.

In relation to performing the pre-assessment and/or onboarding process, the online system and/or other system components can implement data from devices (e.g., non-survey data). For instance, embodiments of the system can perform pre-assessment with implementation of data from devices including one or more of: electronic health record-associated devices; wearable devices (e.g., wrist-borne wearable devices, head-mounted wearable devices, etc.) for monitoring behavior and activities (e.g., related to physiological/cognitive stress, related to respiration activity, related to sedentary and active states, etc.) of the user; non-invasive torso-coupled devices (e.g., abdominal or stomach sensors configured to detect GI or digestive activity); ingestible smart-pill devices; smart toilet devices and/or other devices for analyzing stool and/or urine samples from the subject; and other devices. Non-survey-derived data can additionally or alternatively include data derived from API access of social networking platforms, other communication platforms (e.g., for extracting social behavior characteristics associated with text, voice, and other communications of the users), location-determining platforms, and/or other platforms, in order to assess social behaviors of the user.

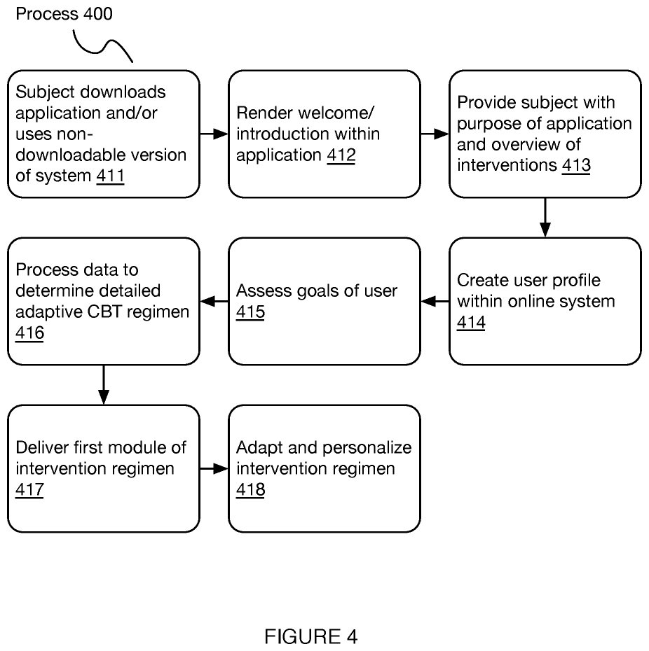

In one example, as shown in FIG. 4, the pre-assessment and onboarding process 400 can include a first step 411 that facilitates downloading of an application and/or using of a non-downloadable version of the system (e.g., via web application, etc.) for delivering the intervention regimen by a client device of the subject; a second step 412 that renders a welcome/introduction screen within the application; a third step 413 that delivers content within the application for educating the subject regarding the purpose of the application and provides an overview of the intervention regimen; a fourth step 414 that creates a user profile within the online system, whereby the fourth step results in a first tier of personalization by implementing survey and non-survey based tools (e.g., to assess gender, age, preferences for scheduling of content delivery, specific GI health condition symptoms of the subject, etc.); and a fifth step 415 that, within the application, assesses goals of the user, whereby the fifth step results in a second tier of personalization. In examples, the second tier of personalization can operate by assessing goals related to anxiety reduction, depression reduction, reduction of IBS and/or IBD or other gastrointestinal disease or syndrome symptoms, improvement of sleep, improvement of socialization, and other goals. In relation to subsequent steps of the method 200, FIG. 4 depicts a sixth step 416 that processes the data from steps 414 and 415 with an intervention-determining model to output a personalized intervention regimen with adaptive cognitive behavioral therapy (CBT) tools and exercises for improving health and wellbeing of the subject, in relation to his/her specific goals. FIG. 4 also depicts a seventh step 417 where a first module of the intervention regimen is delivered to the subject within the application, and an eighth step 418 that provides further adaptation of modules of the intervention regimen as the subject progresses through the intervention regimen and interacts with content.

While the steps of FIG. 4 are shown in a particular order, the steps can be performed in another suitable sequence, omit steps, and/or include additional steps (e.g., based on refinement and training of models described in Section 3.5, based on other factors).

### 3.2 Method—Intervention Regimen and Modules

In relation to system components described above, an embodiment of the online system, in coordination with the network and a client device, can process data from the pre-assessment with an intervention-determining model. Block 220 functions to generate an intervention regimen for the subject upon processing pre-assessment data, in order to design a customized intervention regimen to address specific symptoms and needs of the subject. While Block 220 is described in relation to pre-assessment data, model architecture and associated algorithms can additionally or alternatively be applied to assessment of subject data as the subject interacts with content of the intervention regimen, in order to adaptively modify delivery of intervention regimen components to the subject, with processing of incoming data.

In embodiments, the intervention-determining model contemporaneously processes data associated with user goals, user GI health symptoms, user mental health states, other characteristics, and interactions with content of application providing the intervention regimen as inputs, in order to output a customized and modulatable intervention regimen to improve the health and/or wellbeing of the subject. The intervention-determining model can include architecture for one or more of: conditional decision making (e.g., with conditional branching structure that processes input data in stages and determines an output at each node of the branching structure); ranking (e.g., with ranking algorithms configured to rank candidate intervention regimen components according to appropriateness, based on the input data); matching (e.g., with performance of best match operations between input data and different groups representing modules of the intervention regimen, with centroid-based approaches, etc.); correlation (e.g., correlation functions that process input data to generate outputs associated with different intervention regimen components); and/or any other suitable architecture. Training of models is further described below.

The online system, in coordination with other system components (e.g., the client device, external systems, network, etc.) then delivers 230 the intervention regimen to the subject, for instance, through an application executing at the client device of the subject.

As described in relation to the system above, content associated with the intervention regimen can be of visual (e.g., image format, video format), textual, audio, haptic, and/or other formats, through connected devices (e.g., mobile computing devices, wearable devices, audio output devices, displays, temperature control devices, lighting control devices, etc.) and generated in a manner that promotes user engagement. Furthermore, the system, in providing the interventions (e.g., such as interventions described in more detail below), can coordinate with and/or provide instructions for control of other devices, for intervention delivery. In variations, the system can coordinate with environmental control devices (e.g., connected audio output devices, connected temperature control devices, connected lighting control devices, connected pill dispensing devices, connected smart pill devices, etc.) to change aspects of the subject's environment in association with provision of the intervention regimen.

In one example of an intervention regimen component for reducing anxiety, the intervention regimen can provide a grounding exercise to reduce anxiety regarding GI health condition symptoms, where the user is prompted to observe aspects of the environment with multiple senses, and the system can coordinate with environmental control devices to adjust one or more of lighting (e.g., colors, intensity, etc.), sounds (e.g., through audio output devices), and/or temperature in the subject's environment. In another example, the intervention regimen can provide a relaxation exercise to reduce pain associated with GI health condition symptoms, and coordinate with an audio output device to play music pleasing to the subject. In another example, the intervention regimen can provide an exercise activity involving movements or dancing, to reduce bloating and depression associated with GI health condition symptoms, and coordinate with an audio output device to play dance music to the user, while reducing environmental temperature with a smart thermostat device. The system can provide coordinated interventions, however, in any other suitable manner, where details of interventions are provided in more detail below.

### 3.2.1 Intervention Types and Details

In variations, the intervention regimen provides, through client devices, an array of empirically-supported intervention options or actions delivered via a modular and flexible approach, whereby modules of the regimen (a set of overarching principles and evidence-based interventions) can be adaptively provided based on patient states assessed in real-time or near real-time. This allows for individualized treatment planning.

The order of modules of the intervention regimen provided can vary from patient to patient and/or vary based on other factors (e.g., due to refinement and training of models, as described in Section 3.5); however, in some embodiments, all patients will have access to and be offered all of the skill modules through applications executing on their respective client devices. The skills-based interventions rely on skill acquisition (initial phase of learning the new skill), then skill practice before proceeding to learn the subsequent new skill (e.g., in one's natural home/social environment), where monitoring of task performance and practicing skills is described with respect to Block 240 below. In particular, the modules can allow users to develop and train core skills (e.g., 8 core skills, another suitable number of core skills, etc.) associated with understanding their disease and/or condition, therapies available, brain-gut connections; relaxation skills; behavioral change, avoidance, and activation; problem solving and coping; pain management; cognitive flexibility; social problem solving and communication; and relapse prevention and skills maintenance.

Disease, condition, and/or syndrome-specific components include content addressing one or more of: an illness narrative, symptom management for pain and other symptoms, disease-specific psychoeducation, social skills training, and emphasis on GI health condition (e.g., IBS-related, IBD-related) cognitions, beliefs, and behaviors. Intervention modules can further include general cognitive behavioral components shared across psychological conditions/disorders such as behavioral activation, attentional processes, relaxation, problem solving, cognitive reframing, and other areas.

With respect to mechanisms of action, the behavioral and cognitive change interventions described below interrupt the problematic behaviors that are maintaining/perpetuating the targeted symptoms, provide new adaptive coping strategies, and improve perceived control of symptom management in a positive manner. Furthermore, with the adaptive intervention design, the ability to tailor ‘at the right time’ requires relevant information about the user that is used to decide under what conditions to provide an intervention and the appropriateness of the intervention.

Introduction and Education Module—In a variation of the intervention regimen, an introduction and education module focuses on education about the subject's disease (e.g., IBD, etc.) and symptoms (e.g., more common symptoms, less common symptoms, etc.), provides information regarding methods of diagnosis, promotes understanding of functional implications of symptoms in the context of brain-gut axis education (e.g., with effect to the brain's role in gut motility, secretion, nutrient delivery, and microbial balance, and the gut's role in neurotransmitter dynamics, stress and anxiety, mood, and behavior), creates awareness about what matters to the subject (their reason for trying the program), introduces therapy concepts (e.g., related to CBT, related to other therapies), introduces skills that the user will build by interacting with the system, and assesses user's level of commitment for change.

An overview of this program links to the patient's specific psychological/disease management challenges. The following points are emphasized: (1) the treatment is modular/flexible in nature and tailored for subject's needs (2) the subject will learn skills, that if practiced, will help them manage their symptoms (e.g., with highlighting of red flag symptoms), improve their quality of life, and lessen the toll that IBS, IBD, or other GI conditions, takes on the subject. This module thus can guide the subject to explore the influence that moods, attitudes, beliefs and behavior exert on health and the impact of illness. This module can further function to provide tools for education, persuasion (e.g., regarding effectiveness of program completion), personalization, motivation enhancement, setting expectations, eliciting commitment by users, and establishing a relationship between users and the system (e.g., in lieu of a human coach, with supplementation of therapy by a human coach, etc.). Delivery methods for this module can include one or more of: graphics/animations depicting gut-brain connections, metaphorical digital content, interactive exercises provided in an application environment, and a clinical vignette simulating patient-provider interactions.

In a specific example, the introduction and education module includes a First Section configured to welcome the subject and introduce the subject to goals of the intervention regimen delivered through the online system and client device. The First Section is delivered by the system in an interactive format (e.g., with video and text content) that creates a feedback loop with users and processes user responses to tailor subsequent module delivery and content, in order to increase engagement. As such, goals can be set in coordination with user desires, with establishment of collaborative empiricism. Goals can be specific, in terms of detailed planning of what users will do, including frequency, intensity, duration, and context (e.g., where, when, how, with whom, etc.) of the goal(s). Furthermore, in relation to interactive content, the introduction and education module can determine topics having greater relevance to the user's current issues (e.g., in relation to comorbid conditions, such as anxiety and depression, in relation to GI health condition subtypes, such as subtypes of IBS, etc.). In variations of the method 200 configured for regular use (e.g., at a frequency more often than weekly interaction), the First Section can include a description of how the program will involve regular practice (e.g., daily, every two days, every 3 days, etc.) of skills (e.g., core skills described above and below), with a guideline for program length (e.g., 8 weeks, less than 8 weeks, more than 8 weeks), and methods of identifying personal progress (e.g., feeling better with mastery of a subset of skills).

In the specific example, the introduction and education module includes a Second Section configured to allow the subject to submit information, through the application, regarding personal aspects of his/her GI health condition (e.g., IBD) as an initial physical illness narrative, along with video content to which the subject can compare his/her experiences. The Second Section has goals of facilitating emotional awareness, establishing a physical illness narrative that can be revisited as the user gains mastery of skills, and helping the user to articulate and track his/her experiences.

In the specific example, the introduction and education module includes a Third Section configured for personalization of subsequent portions of the intervention regimen to the subject, by allowing the subject to indicate, through the application, which symptoms (e.g., fatigue, pain, nausea, vomiting, lack of appetite, weight loss, skin problems, eye problems, joint problems, diarrhea, bowel movement issues, cramping pains, bloody stool, medication side effects, other symptoms, etc.) are most bothersome. The Third Section can also include architecture for mapping the user's symptoms and GI health condition-induced factors to various impacts associated with the user's values. In variations, one or more of the following mappings can be created: symptoms associated with diarrhea, abdominal pain, urgency, tenesmus, nocturnal bowel movements, rectal bleeding, physical fatigue, and other physical symptoms with mappings to aspects of life (e.g., relationships, work, school, hobbies, daily activities, etc.) that have been affected by such symptoms and the reason such aspects have been affected; medication side effects with mappings to aspects of life (e.g., relationships, work, school, hobbies, daily activities, etc.) that have been affected by such symptoms and the reason such aspects have been affected; social/relationship issues (e.g., stress on loved ones, impacts on friendships, etc.) with mappings to behaviors (e.g., relationships, work, school, hobbies, daily activities, etc.) that have been affected by such symptoms and the reason such aspects have been affected; and behavioral, mental, and emotional factors (e.g., exhaustion, lack of control, inability to perform activities, additional help needed for tasks, limitations in diet, limitations in travel, embarrassment, worry, disease progression, lack of confidence, dwelling thoughts, etc.) with mappings to aspects of life affected and lessons learned with onset of state change.

The Third Section has goals for providing education about GI health condition symptoms and psychological consequences (e.g., behavioral psychological consequences), as well as generating data for future personalization of the intervention regimen.

In the specific example, the introduction and education module includes a Fourth Section configured for personalization and values identification, with tools for allowing the user to provide data related to positive and negative changes in his/her life that are attributed to having the GI health condition (e.g., IBD), in relation to changes in relationships, levels of embarrassment, curiosity, being understood, stress to self and loved ones, confidence, energy levels, senses of lack of control, worry (e.g., about GI issues experienced outside of a comfortable environment, about disease progression, and symptoms, about medication effects, about ability to conduct daily activities, about dietary constraints, about travel, etc.), and other aspects. The Fourth Section can also revisit aspects of the user's physical illness narrative, with ranking of: symptoms (e.g., physical fatigue, abdominal pain, diarrhea, urgency, tenesmus, bowel movements at night, rectal bleeding, medication side effects, etc.); social/interpersonal factors (e.g., changes to relationships, embarrassment, stress to loved ones, dealing with constant questions about illness, not being understood, etc.); emotional factors (e.g., lack of confidence, mental exhaustion, lack of control, etc.); cognitive factors (e.g., worry about GI issues outside of places of comfort, worry about GI progression, catastrophizing, depression, anxiety, other comorbid conditions, etc.); and behavioral factors (e.g., not being able to conduct daily activities, needing to prepare for accidents, dietary restrictions, travel restrictions, etc.).

In the specific example, the introduction and education module includes a Fifth Section configured for allowing further customization, by providing the subject with interactive elements that allow the subject to prioritize the order in which content associated with interventions is received.

In the specific example, the introduction and education module also includes a Sixth Section configured for introducing subsequent portions of the intervention according to user preferences indicated from outputs of the Fifth Section, where the goals of the Sixth Section include promotion of treatment credibility (e.g., through presentation of video content by patients having experiences similar to those of the user(s)).

In the specific example, the introduction and education module includes a Seventh Section configured for delivery of content for educating the subject about the Brain-Gut connection, where the content includes an animated element and audio format content configured to actively interact with the user. The interactive elements function to gauge how well the subject understands the content provided, and to provide additional content to engage and inform the subject depending upon responses of the subject. The Seventh Section has goals of shaping knowledge of symptoms and treatment components of the intervention regimen and enhancing motivation.

In more detail, the seventh section can teach users of the system regarding the brain's role in proper gut functioning, and the connection between the mind and the gut. As such, the user can be primed to gain skills related to affecting gut functioning and regulation by changing behaviors, attentional biases, and automatic thought patterns. This section can further gage internalization and understanding of the user, with provision of further content in this section and/or the eighth section to promote further understanding.

In the specific example, the introduction and education module includes an Eighth Section configured for delivery of content for educating the subject about the Brain-Gut connection in a manner personalized to the subject, where the content includes video and audio format content configured to actively interact with the user, in order to aid the user in understanding how the Brain-Gut connection can influence perception of symptoms, based on symptom severity (e.g., related to a threshold level of severity of symptoms, related to fight-or-flight responses, related to gut bacteria and roles in affecting symptoms, etc.). The Eighth Section also provides interactive exercise for learning about physiological-cognitive pathways for perceiving and responding to experienced symptoms and implements architecture for assessing stress and other disease aspects, with implementation of CBT-based techniques for changing reactivity of the brain, thereby decreasing symptom severity and promoting regulation of GI functioning.

In the specific example, the introduction and education module includes a Ninth Section configured for eliciting commitment from the subject, in relation to different set goals of the subject. The digital content of the Ninth Section includes interactive elements for creating a reminder system (according to personalized user preferences and formats for receiving reminders), and interactive elements for setting goals to improve one or more aspects of dealing with the subject's health condition (e.g., with a menu of choices as well as a field for custom user inputs and a field for prompting the user to confirm chosen goals, where example choices can include repeating of tasks, reviewing content, reflecting, identifying entities for social accountability, relocation of application icons on a home screen of a device in a manner that promotes regular use, identifying factors that may obstruct progress, etc.), where the interactive elements allow the subject to confirm when (e.g., specific times), how often, and where the subject will perform activities to meet such goals. The interactive elements further include fields for allowing the subject to set “plan B” options in the event the subject faces obstacles for meeting goals. Finally, the Ninth Section includes a brief introduction to subsequent modules of the intervention regimen that are customized to the subject. The Ninth Section has goals including setting of expectations, promoting therapeutic persuasiveness, eliciting commitment, increasing user engagement, providing reminders, providing instruction for performing behaviors (e.g., SMART goals).

While the sections are described in a particular order above, variations of the introduction module can additionally or alternatively be arranged in another suitable order, omit sections as desired, and/or include additional sections as desired.

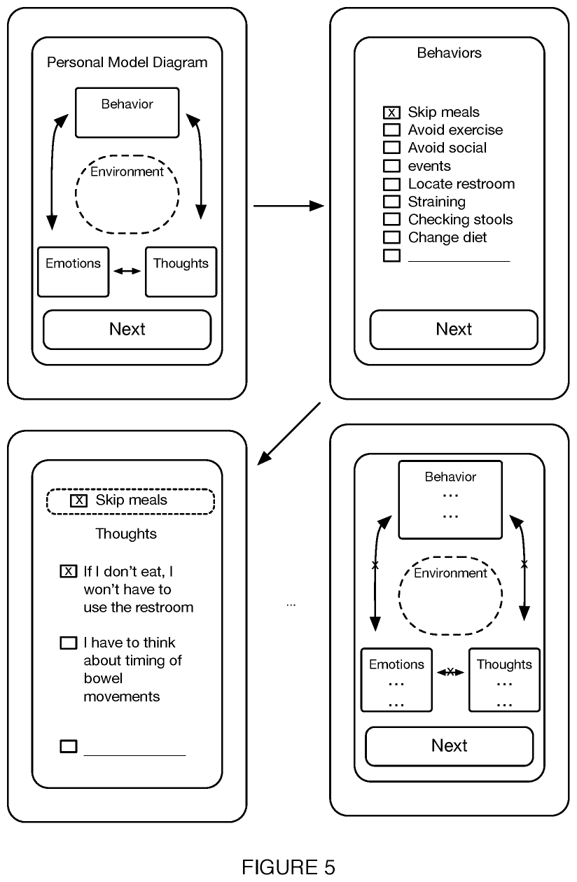

Physical Illness Narrative Module and Symptom Assessment—In a variation of the intervention regimen, a physical illness narrative module provides a form of validation (being heard), highlights cognitive distortions/attentional biases and other clinically relevant processes to address, as well as begins the work of emotional exposure. It also provides a point of reference for reflection throughout and at the end of the program. This module promote formation of a personal disease model for users, such that they can identify patterns in their disease expression and/or progression, in relation to biology, behaviors, environment, emotions, and thoughts (an example of which is shown in FIG. 5). Key functions of this module can include validation of a patient's experience, enhancement of self-understanding and illness comprehension, setting the stage for application of CBT skills to accept uncontrollable elements of physical illness and/or or increase proactivity to address controllable elements of physical illness, and generation of interest for patient engagement.

In an example, the physical illness narrative module can receive user report data (or other data) regarding the user's illness history (e.g., painful experiences in a clinical setting, such as with a clinician or hospital environment), thoughts (e.g., thoughts of guilt or responsibility for condition and behaviors, etc.), emotions (e.g., in relation to helplessness, feeling worthless, in relation to embarrassment, etc.), in order to address cognitive distortions for emotional exposure throughout subsequent interactions with the system.

Additionally or alternatively, the physical illness narrative module can include architecture for prompting the user to provide data and/or automatically receiving data (e.g., through API access of health monitoring applications, through receiving of sensor signals of devices of the user, etc.) pertaining to one or more of: pain symptoms, stress symptoms, diarrhea and stool aspects, accidents incurred, constipation and stool aspects, amount of time straining, meals eaten/skipped and times of meals, behaviors and behavioral changes, and other aspects.

Additionally or alternatively, as shown in FIG. 5, the physical illness narrative module and/or other related modules can include architecture for prompting the user to provide data and/or automatically receiving data (e.g., through API access of health monitoring applications, through receiving of sensor signals of devices of the user, etc.) pertaining to one or more of: biological aspects (e.g., physiological symptoms); behavioral aspects (e.g., in relation to skipping meals, in relation to exercise avoidance, in relation to social event behavior, in relation to locating restrooms, in relation to straining, in relation to checking stools, in relation to other aspects); environmental aspects (e.g., in relation to stress, in relation to temperatures, in relation to diet, etc.); emotional aspects; and thoughts linked to behaviors (e.g., regarding anxiety around diet, regarding to anxiety around performing various activities, etc.), and automatically return an analysis summarizing the personal model of the user (e.g., in a visual format, etc.). Such personalization thus promotes interruption of vicious cycles for users. As such, in relation to determining characterizations of the GI condition of the user in a personalized manner, the method 200 can include returning a mapping with a network of flows between a set of behaviors specific to the user, a set of thought patterns specific to the user, a set of physiological symptoms specific to the user, a set of emotions specific to the user, and environmental triggers specific to the user, where returned outputs of models described can be configured to disrupt flows of the network contributing to deterioration of symptoms of the user.

Delivery methods for this module can include audio format content and/or textual content for guiding exercises. The physical illness narrative module may be a subcomponent of multiple modules, such that its content can be revisited. For instance, upon development of core skills associated with the modules, the system can trigger revisitation of aspects of the physical illness narrative module (e.g., within the mobile application, within the web application, etc.), such that users can solidify new skills, reflect on their initial versions of their physical illness narrative and what has changed, generalize skills, maintain skills, and implement cognitive flexibility.

Relaxation Module—In a variation of the intervention regimen, a relaxation module provides understanding of what physiological stress feels like (e.g., with education on fight or flight responses) and recognition of the importance of actively optimizing their stress response, particularly because of the connection between stress reactivity, stress hormones and autonomic arousal, and flares in symptoms. The module informs the subject that (1) stress is a natural reaction and it causes its own physical symptoms (2) the brain does not differentiate between an event that is actually happening to us and an event that we only think is happening, and (3) the connection between stress and flares and symptoms. The module provides a rationale for each type of relaxation and how it is tailored for their specific stress symptoms, and provides guided relaxation exercises (e.g., within the application executing at the client device). The module promotes mastery of at least one relaxation technique. Key functions of this module can include decreasing physiological reactivity associated with stress, worry, anxiety, and pain, activation (for depression symptoms), and stress management. Delivery methods for this module can include audio format and/or visual content for guiding exercises associated with targeted muscle groups for progressive muscle relaxation, video-guided demonstration of diaphragmatic breathing, and haptic feedback for exercise guidance.

In a specific example, the relaxation module can include video format content that introduces the general concept of relaxation; educates the subject on the applicability of stress-reduction exercises to GI health conditions, with active text boxes that promote user engagement and personalization of the module to the subject's specific symptoms and contexts; addresses common doubts or concerns about relaxation; promotes a guided breathing exercise with a diaphragmatic breathing demonstration and corresponding animated graphic; promotes guided exercises for muscle relaxation using progressive muscle relaxation (PMR) techniques using graphical animations (e.g., of targeted muscle groups); provides information on how relaxation practices can be used (e.g., for abdominal pain, for anxiety, for other stressors, etc.), and encourages practice of exercises by including active interactive elements that the user can use for scheduling and/or accountability in practicing exercises.

Behavioral Change, Avoidance, and Activation Module—In a variation of the intervention regimen, a behavior change/behavioral activation module provides content covering the importance of activation and approaching avoided situations/experiences in breaking the cycle of persistent pain symptoms and depressive and/or anxious mood. Specific action plans are developed for decreasing avoidance behavior. Key functions of this module can include linking behaviors and mood, mood monitoring (e.g. self-monitoring), activity scheduling, identifying and counteracting avoidance behavior, action planning, activity scheduling, creating anxiety hierarchies, self-monitoring, behavioral experiments, exposure (e.g., imaginal exposure, actual exposure to counteract anxiety) and systematic desensitization for anxiety, coping performance, confidence building, and routine building. Delivery methods for this module can include use of automated tailoring for choosing topics that have greater relevance to patient's current problems (e.g., if a user reports anxiety, information about physiological responses of anxiety and their relationship with thoughts and behaviors would be more appropriate than information about the physiological symptoms of depression or generic stress).

Problem Solving and Coping Module—In a variation of the intervention regimen, a problem solving and coping module provides content covering how to differentiate controllable vs. uncontrollable stressors, problem-focused coping (e.g., with problem identification, solution brainstorming, evaluation of solution options, etc.) vs. emotion-focused coping (e.g., with grounding exercises), as well as types of adaptive and maladaptive coping. Mood/anxiety/stress may be managed/ameliorated by using externally-focused coping to distressing and modifiable conditions and internally-focused coping to adjust one's expectations and interpretations for unmodifiable conditions. In examples, the system can include architecture and instructions for promoting practicing of problem solving and coping methods by the user, such that the user is better able to handle stronger symptoms (and milder symptoms).

Delivery methods for this module can include digital content with explanations and testimonials of other subjects and their uses of problem solving skills, peer support groups facilitated by the application, and other delivery methods.

Pain Management Module—In a variation of the intervention regimen, a pain management module focuses on awareness of the pain experience, discusses how pain influences mood and vice versa, promotes recognition of certain behaviors (e.g., overactivity, avoidance) and automatic thoughts that may influence pain as well as how to feel more in control of pain by also improving physical and role functioning though increasing adaptive behaviors/coping (attention) and decreasing avoidance/maladaptive behaviors. Key functions of this module can include behavioral experimentation, behavior substitution, acceptance of pain, and self-monitoring, with a disease- or syndrome-specific target of abdominal pain. In specific examples, the pain management module can include architecture and content for educating users regarding re-directing attention away from pain symptoms by focusing on parts of the body that are not in pain, and other methods. In more detail regarding this example, the system can include a processor with instructions stored in non-transitory media that when executed, perform steps for identifying when a user is in a state of pain, and triggering a response (e.g., verbal cues and instructions to modify attention and/or engage in various pain observation exercises, a change in the environment of the user, by playing music, by activating a display and providing video or image content, by providing haptic stimulation to the user, etc.).

Delivery methods for this module can include audio format content and/or textual content for managing pain (e.g., with music, exercise, etc.) and/or for promoting attention reconstruction.

In a specific example, the pain management module can include a first Section that includes content focused on common types of pain (e.g., abdominal pain) associated with the user's GI health condition (e.g., IBD, IBS).

In the specific example, the pain management module can include a second Section focusing on facts about chronic pain associated with the user's GI health condition (e.g., IBD, IBS), in relation to constant pain, flare ups of pain, pain signals for people with GI health conditions vs. without GI health conditions, factors affecting pain strength, and other factors. The Second Section can also include image and video content (e.g., including testimonials of patients similar to the user) and other interactive exercises.

In the specific example, the pain management module can include a third section describing differences between acute pain and chronic pain associated with GI health conditions, and therapies associated with each type of pain.

In the specific example, the pain management module can include a fourth section focused on pain volume attributed to specific nerves of the brain, with interactive exercises and content for re-training the brain to adjust pain volume (i.e., pain modulation). The specific example can also include a fifth section focusing on factors that affect pain intensity/perceived pain intensity (e.g., loss of sleep, tense muscles, anxiety, worry, etc.) and methods for modulating pain intensity and duration (e.g., relaxation, distraction, positivity, exercise, medicine, etc.).

In the specific example, the pain management module can include a sixth section describing the importance of relaxation in modulating pain volume and creation of a pain management plan.

In the specific example, the pain management module can include a seventh section focused on the effects of pain on negative emotions, with architecture for including customized content from the user's illness narrative (associated with other modules), in a textual, audio, and/or visual format, and allowing the user to update his/her illness narrative.

In the specific example, the pain management module can include an eighth section focused on development of automatic habitual thinking patterns to interrupt and break these negative cycles. The specific example can also include a ninth section with architecture for presenting a patient testimonial regarding a personal experience of catastrophizing thoughts and the effects on worsening mood, pain, and perpetuation of biased attentional processing.

In the specific example, the pain management module can include a tenth section focused on promoting a healthy lifestyle to protect the body against stress, pain flares, and other GI health condition symptoms. In the specific example, the pain management module can include architecture for helping the user establish goals in various activities in his/her daily life (e.g., school, friendship, sports, etc.), as they relate to pain management.

In the specific example, the pain management module can include a twelfth section focused on activity pacing to prevent increases in pain, with interactive content (e.g., derived from patient testimonials, etc.). The specific example can also include a thirteenth section focused on providing examples of activity pacing (e.g., taking breaks during physical exercise, setting limits in relation to pain thresholds, etc.), with interactive modules for setting goals specific to activities that the user values and/or enjoys.

The specific example of the pain management module can also include a fourteenth section focused on helping the user to generate a pain management plan with respect to relaxation skills gained (e.g., diaphragmatic breathing, progressive muscle relaxation, etc.), cognitive flexibility skills (e.g., catastrophizing avoidance, etc.), eating and drinking habits (e.g., with respect to regular meals with respect to caffeine limitation, etc.), with respect to activity performing, and with respect to activity pacing.

Cognitive Reconstruction and Flexibility Module—In a variation of the intervention regimen, a cognitive flexibility module targets one's interpretations of events/experiences (e.g., how core thoughts influence our feelings and behavior). This module emphasizes connections between thoughts and physical sensations due to GI symptoms. The aim of this module is to teach patients how to identify unhelpful automatic thinking patterns and develop a new pattern of realistic, balanced, and flexible thinking. A health behavior change is targeted in the area of sleep and worry by providing education about worry and how it might interfere with sleep. Strategies to manage worry before bedtime (e.g., use a relaxation practice) are provided as well as basic sleep hygiene. Key functions of this module can include resetting of cognitive distortions (e.g., about self, others, and the world), identification of unhelpful thoughts, challenging of automatic thoughts, creating more balanced thoughts, re-attribution, appraisals of moods, and improving cognitive flexibility. Delivery methods for this module can include a tool providing digital content for reassembling a traditional thought record in which patients enter an unhelpful automatic thought and select from a list of negative thoughts that best matched. After selecting from list of most common automatic thoughts, the tool can generate a list of possible challenge/alternative thoughts. The subject can then input their own personalized challenge/alternative thought.

Social Problem-Solving, Social Skills, and Social Support Module—In a variation of the intervention regimen, a social problem-solving, social skills, and social support module provides content promoting effective social behaviors in the context of GI health conditions. The social problem-solving, social skills, and social support module can provide tools for one or more of: action planning, social skills training, social support, exposure, and activation, with identification of oneself as a role model, and presentation of information regarding vicarious consequences. In relation to disease- or syndrome-specific targets involving social problem-solving, this module is intended to assist interactions between subjects and their social environment in the context of their GI health condition(s), and how to communicate effectively about the medical condition/disease. Some examples include requesting support in college (disability services office) or at work; informing a subject that his/her behavior may be an example to others; coping with sense of urgency to use bathroom; and problem solving about bathroom/bowel related challenges and worries. Key functions of this module can include activation and action planning, problem solving by analysis of factors influencing the behavior and generating strategies to overcome barriers, demonstrating one's ability to cope, decreasing avoidance behaviors, ensuring practice of new coping skills, when symptoms are more severe (e.g., with behavioral rehearsal, etc.). Delivery methods for this module can include digital content with testimonials of other subjects and their uses of problem solving, peer support groups facilitated by the application, and other delivery methods.

In examples, this module can include architecture for triggering actions based on detected changes in symptoms. For instance, in one example, this module can process data generated by interactions between the user and the system (e.g., with sensor-based monitoring of symptom progression, with user input-based monitoring of symptom progression, etc.), and based upon the data, generate control instructions for recommended actions that would improve social problem solving ability. Examples of recommended actions can include one or more of: guidance for conducting a conversation regarding symptoms (e.g., example language for communicating pain, defecation, or other-related symptoms to an entity, so that the user can experience relief, etc.); triggering automatic communications between the user and an entity (e.g., automatically sending a private message to a teacher so that the teacher can excuse the user to manage pain-related, defecation-related, and/or other symptoms); and performing other suitable actions.

Relapse Prevention and Skills Maintenance Module—In a variation of the intervention regimen, a relapse prevention and skills maintenance module encourages maintenance/continuation of treatment gains, and reinforces positive changes in thoughts and behavior that were accomplished during the active treatment time. Key functions of this module can include skills generalization, skills maintenance, and adaptive monitoring to refresh skills learned. Additionally, this module can perform one or more of: informing users of signs of relapse into old patterns, development of specific proactive coping tools for future challenges, encouragement of proactive coping for mood regulation, explaining perseverance, education regarding sequential coping strategies, and identification of skills/techniques that were most effective for the user, based on analysis of user outcomes. Delivery methods for this module can include digital content and/or notifications related to monitored states of the subject (e.g., related to relapse) as described further in relation to Block 240 below.

Examples of Behavioral Performance Tasks and Assessments—In examples, exercises associated with the intervention regimen can include one or more of: a card sorting task to identify user's reinforcers/motivators (e.g., in relation to social reinforcers, reminders, accountability, gaming/competition, responsiveness to quantitative summary feedback, monetary incentives, altruism, learning, elimination of symptoms, etc.); computerized performance tasks (e.g., delayed discounting) to measure/identify salient reinforcers and/or learning style; and performance tasks (e.g., validated distress tolerance computer tasks, tasks associated with mimicked social interactions, etc.) to measure emotional awareness and ability to tolerate various types of distress (psychological, physical, etc.). Aspects of the examples and variations described can be implemented in coordination with performing a subject pre-assessment (e.g., in relation to non-survey data used for assessments), as described above.

Additional or Alternative Interventions—While some intervention types and associated content are described above, Blocks 220 and 230 can include delivery of other interventions, by way of the online system in coordination with other devices, where monitoring of performance of activities with such interventions is described below. Such interventions can include one or more of: anti-inflammatory pharmacologic therapies (e.g., 5-aminosalicylic acid derivatives), corticosteroids, immunomodulators, biologics, nutritional therapies (e.g., enteral nutrition), natural products, whole system medicine (e.g., Eastern Medicine, Ayurveda), mind-body interventions (e.g., yoga, clinical hypnosis), psychotherapy, acceptance and mindfulness-based therapies, biofeedback (e.g., for control of the autonomic nervous system, for control of the cardiovascular system) using biofeedback devices for treating abdominal pain and other symptoms, and other interventions that can be delivered using associated devices.

While the modules are described in a particular order, the modules can be performed in another suitable sequence, omit steps, and/or include additional steps (e.g., based on refinement and training of models described in Section 3.5, based on other factors). Furthermore, aspects of the modules can overlap with each other in any suitable manner.

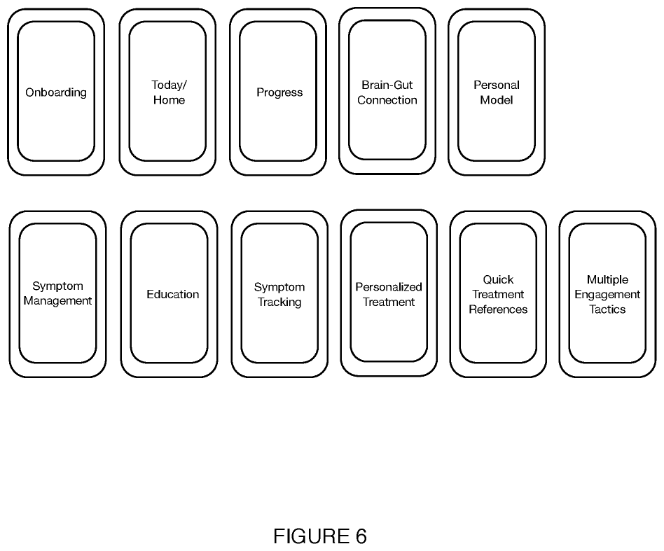

FIG. 6 depicts an example of module components delivered through an application (e.g., mobile application, web application), where content includes: onboarding material, daily (or other time scale) review, progress summaries, brain-gut connection content, personal model analyses, symptom management material, educational material, symptom tracking analyses, personalized treatment analyses, quick references, and multiple engagement tactics material.

### 3.2.2 Example Intervention Regimen Pathways

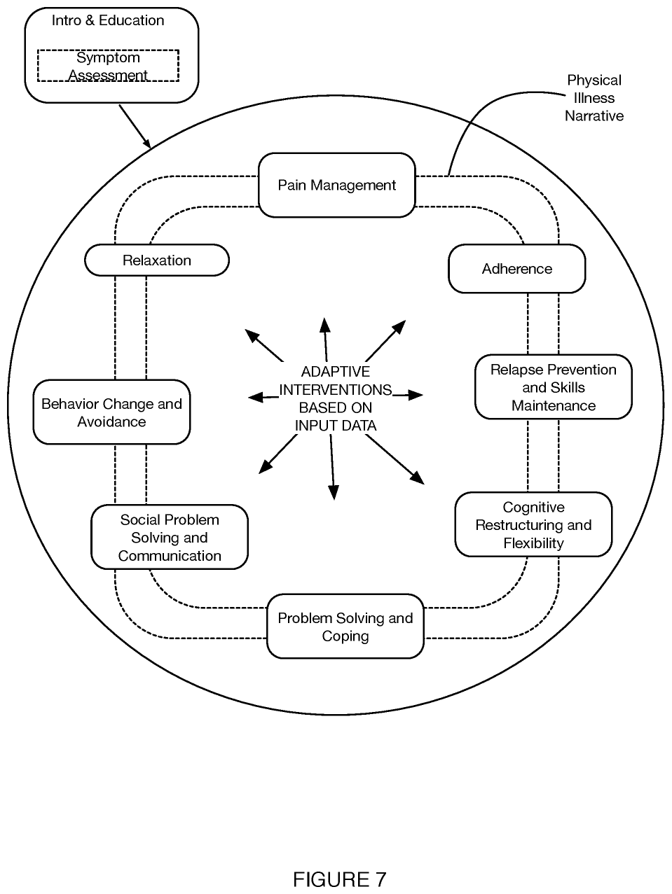

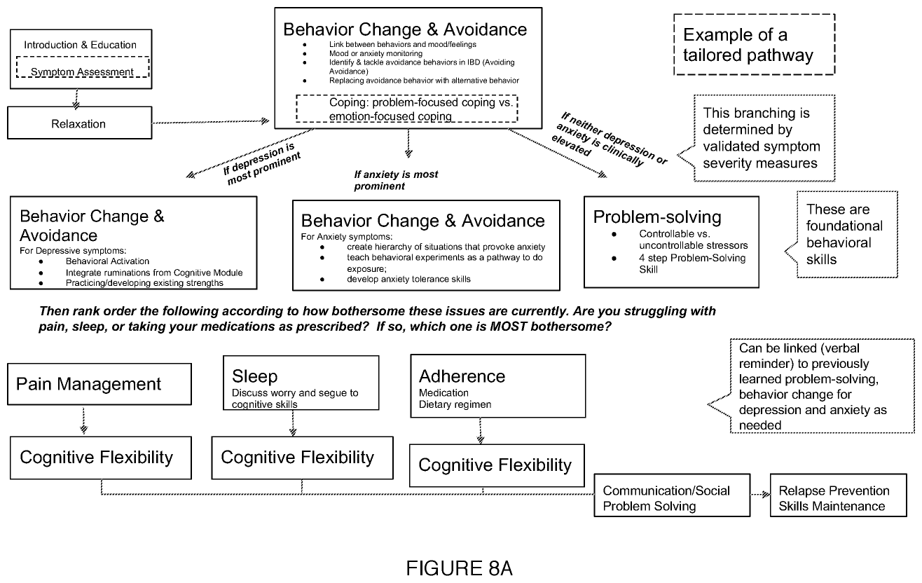

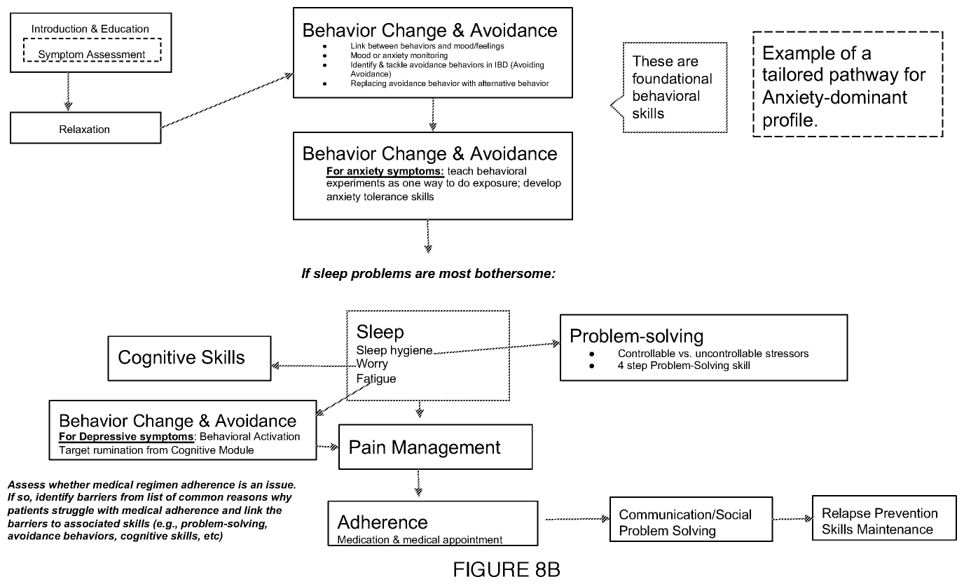

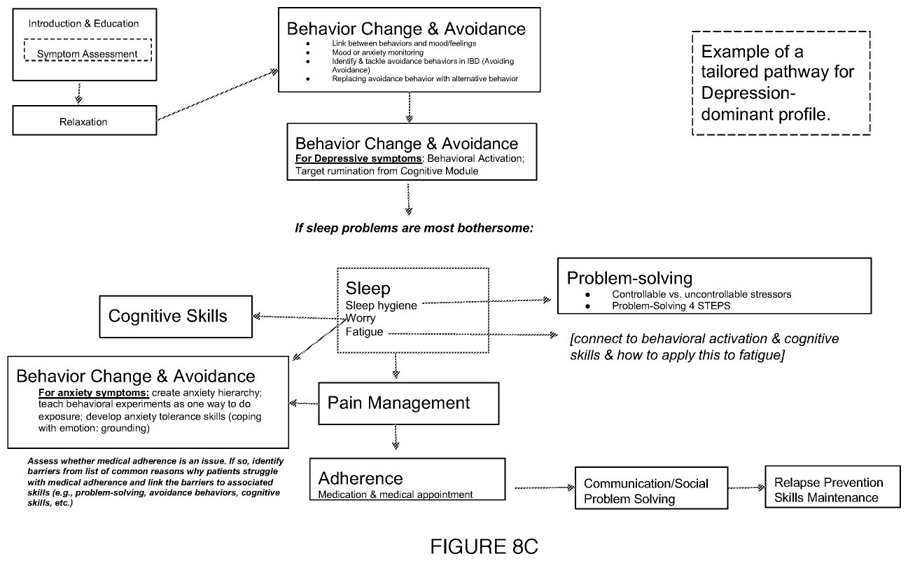

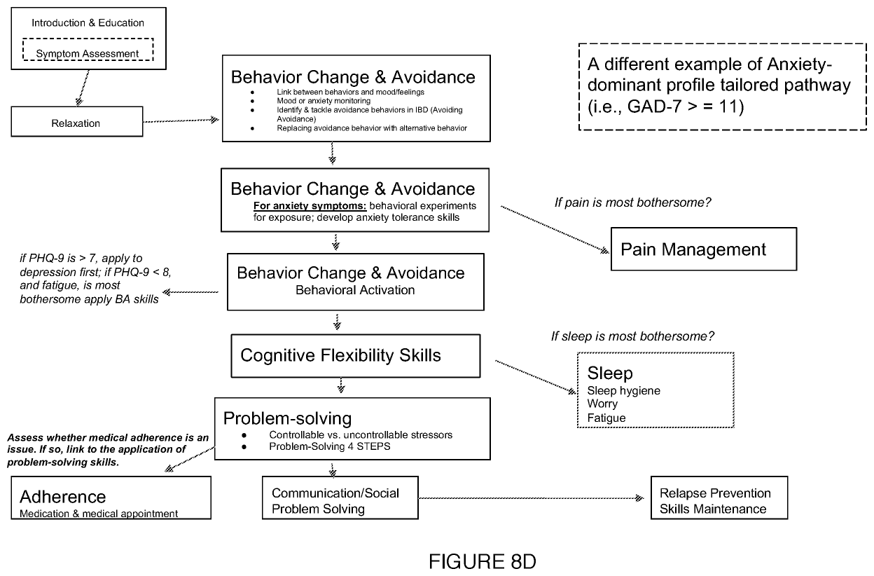

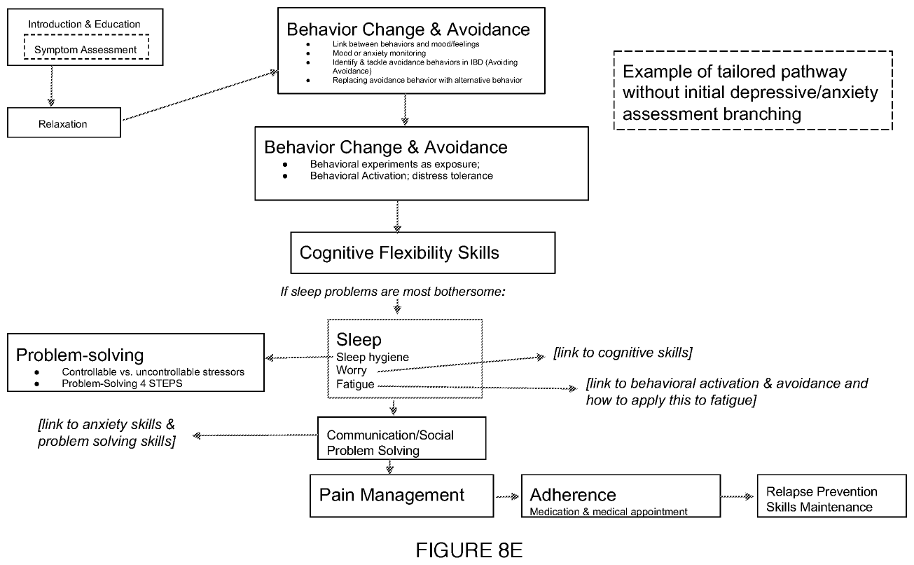

FIG. 7 depicts a flow chart of example adaptive intervention regimen pathways shown in FIGS. 8A-8E.

In more specific examples, as shown in FIGS. 8A-8E, the intervention-determining model includes architecture for processing input data (e.g., from the pre-assessment and in real-time as the subject interacts with content of the intervention regimen), with a conditional branching model (e.g., with if-then branches coupled to nodes associated with outputs) that processes input data to tailor individual psychological interventions to the subject in an individualized manner. The conditional branching model thus includes decision rules linking characteristics of the subject (e.g., clinical and symptom presentation, demographics, etc.) to different components of the intervention regimen, as an adaptive intervention.

FIG. 8A depicts architecture of the conditional branching model for a generalized pathway where, based on severity of physical illness symptoms exhibited by a subject, the model guides (e.g., through the application executing at the client device) the subject through foundational behavioral skills appropriate to the state and goals of the subject. After a core set of skill building exercises is provided to the subject, the order of modules can vary from subject to subject. Decisions (within app) about which modules to prioritize first are based on patient's presentation and needs (e.g., symptom patterns, etc). For example, if abdominal pain is what is most bothersome to the patient, the digital therapeutic will recommend the pain management module after completing one of the modules (e.g. the relaxation module).

In more detail, based on demonstrated symptoms, the conditional branching model shown in FIG. 8A selects a Behavior Change and Avoidance module for delivery, where the module informs the subject of links between behaviors and moods/feelings, and actively coaches the subject with respect to addressing avoidance behaviors in relation to GI health condition symptoms, in order to replace avoidance behaviors with alternative healthier behaviors. The CBT techniques implemented in the selected intervention can address problem-focused coping tools and/or emotion-focused coping tools, with additional tailoring for different mental health issues associated with the GI health condition symptoms of the subject. For instance, if the subject's depression is most prominent, the conditional branching model outputs behavioral activation exercises, cognitive reframing techniques, talent practicing and reinforcement exercises, and/or other exercises to mitigate depression symptoms. Additionally or alternatively, if the subject's anxiety is most prominent, the conditional branching model outputs exposure-based exercises associated with anxiety sources, anxiety tolerance skill-building exercises, grounding exercises, and/or other exercises to mitigate anxiety symptoms. Alternatively, if neither anxiety nor depression are elevated, the conditional branching model outputs problem-solving exercises with respect to controllable vs. uncontrollable stressors, and other exercises to mitigate problem-solving issues. The conditional branching model further receives inputs (e.g., rankings of symptom severity) related to symptoms that the subject wishes to improve (e.g., related to pain management, related to sleep, related to adherence, related to communication, related to social problem solving, related to relapse prevention, etc.), and then based upon the inputs, guides the user through additional cognitive skills tailored to improve symptoms in the manner that the subject desires.

FIG. 8B depicts architecture of the conditional branching model for a anxiety-specific pathway where, based on severity of physical illness symptoms exhibited by a subject, the model guides (e.g., through the application executing at the client device) the subject through foundational behavioral skills appropriate to the state and goals of the subject. In more detail, based on demonstrated symptoms, the conditional branching model selects a Behavior Change and Avoidance module for delivery, where the module informs the subject of links between behaviors and moods/feelings, and actively coaches the subject with respect to addressing avoidance behaviors in relation to GI health condition symptoms, in order to replace avoidance behaviors with alternative healthier behaviors. For a subject having prominent anxiety symptoms, the conditional branching model of FIG. 8B outputs exposure-based desensitization exercises associated with anxiety sources, anxiety tolerance skill-building exercises, grounding exercises, and/or other exercises to mitigate anxiety symptoms. The conditional branching model of FIG. 8B further receives inputs (e.g., rankings of symptom severity) related to sleep and/or other symptoms (e.g., fatigue, sleep hygiene, worry, etc.) that the subject wishes to improve, and then based upon the inputs, guides the user through additional cognitive skills, problem-solving exercises, and behavior change exercises, tailored to improve sleep symptoms related to his/her GI health condition.

FIG. 8C depicts architecture of the conditional branching model for a depression-specific pathway where, based on severity of physical illness symptoms exhibited by a subject, the model guides (e.g., through the application executing at the client device) the subject through foundational behavioral skills appropriate to the state and goals of the subject. In more detail, based on demonstrated symptoms, the conditional branching model selects a Behavior Change and Avoidance module for delivery, where the module informs the subject of links between behaviors and moods/feelings, and actively coaches the subject with respect to addressing avoidance behaviors in relation to GI health condition symptoms, in order to replace avoidance behaviors with alternative healthier behaviors. For a subject having prominent depression symptoms, the conditional branching model of FIG. 8C outputs behavioral activation exercises, cognitive reframing techniques, and reinforcement exercises, and/or other exercises to mitigate depression symptoms. The conditional branching model of FIG. 8C further receives inputs (e.g., rankings of symptom severity) related to sleep and/or other symptoms (e.g., fatigue, sleep hygiene, worry, etc.) that the subject wishes to improve, and then based upon the inputs, guides the user through additional cognitive skills, problem-solving exercises, and behavior change exercises, tailored to improve sleep symptoms related to his/her GI health condition.

FIG. 8D depicts architecture of the conditional branching model for a pathway targeted to anxiety and depression (e.g., with a GAD-7 score greater than or equal to 1) where, the model guides (e.g., through the application executing at the client device) the subject through foundational behavioral skills appropriate to the state and goals of the subject. In more detail, based on demonstrated symptoms, the conditional branching model selects a Behavior Change and Avoidance module for delivery, where the module informs the subject of links between behaviors and moods/feelings, and actively coaches the subject with respect to addressing avoidance behaviors in relation to GI health condition symptoms, in order to replace avoidance behaviors with alternative healthier behaviors. For a subject having prominent anxiety symptoms, the conditional branching model of FIG. 8D outputs exposure-based desensitization exercises associated with anxiety sources, anxiety tolerance skill-building exercises, grounding exercises, and/or other exercises to mitigate anxiety symptoms. The model also determines if the subject is suffering from pain symptoms, and provide the subject with pain management exercises. The model also then sequentially determines if the user is exhibiting symptoms of depression (e.g., if PHQ-9 score is greater than or less than 10), and addresses depression symptoms sequentially relative to other symptoms (e.g., sleep, communication, medication adherence) based upon symptom severity.

FIG. 8E depicts architecture of the conditional branching model for a pathway that is not specific to anxiety or depression where, based on severity of physical illness symptoms exhibited by a subject, the model guides (e.g., through the application executing at the client device) the subject through foundational behavioral skills appropriate to the state and goals of the subject. In more detail, based on demonstrated symptoms, the conditional branching model selects a Behavior Change and Avoidance module for delivery, where the module informs the subject of links between behaviors and moods/feelings, and actively coaches the subject with respect to addressing avoidance behaviors in relation to GI health condition symptoms, in order to replace avoidance behaviors with alternative healthier behaviors. For a subject having no initial anxiety/depression symptoms, the conditional branching model of FIG. 8E outputs problem-solving exercises with respect to controllable vs. uncontrollable stressors, and other exercises to mitigate problem-solving issues. The conditional branching model of FIG. 8E further receives inputs (e.g., rankings of symptom severity) related to sleep and/or other symptoms (e.g., fatigue, sleep hygiene, worry, etc.) that the subject wishes to improve, and then based upon the inputs, guides the user through additional cognitive skills, problem-solving exercises, and behavior change exercises, tailored to improve sleep symptoms related to his/her GI health condition.

### 3.3 Method—Monitoring Progress

In relation to system components described above, an embodiment of the online system, in coordination with the network and a client device, can monitor 240 a set of interactions between the subject and modules of the intervention regimen and a health status progression of the subject contemporaneously with delivery of the intervention regimen. Monitoring interactions functions to provide intimate understanding of progress of the subject in achieving health goals, and to provide further personalization of and delivery of intervention content at appropriate times, in order to maintain or improve progress of the subject. Monitoring is preferably performed in near-real time or real time, such that actions can be taken to adjust interventions to user states according to JITAI protocols. However, monitoring can be performed with any suitable delay (e.g., in relation to achieving better accuracy of assessed states of the subject).

Monitoring can be performed using survey components delivered with interactive interventions of the intervention regimen, where the user is prompted and provided with interactive elements that allow the subject to provide self-report data indicating progress statuses. Monitoring can additionally or alternatively be performed with processing of other data streams, where the data streams are associated with application or device usage metrics, social networking behavior extracted from usage of social networking applications and communication applications, sensor-derived data, and/or other data. Monitoring can thus occur with any frequency and/or level of intrusiveness.

In variations, Block 240 can process data (e.g., real time data, non-real time data, dynamic data, static data) with a predictive model that outputs indications of one or more of symptom severity predictions, predictions of subject states, indications of predicted success of the subject in achieving goals, and/or other predictions, where training of the predictive model with training sets of data is described in Section 3.5 below.

In a variation, ecological momentary assessments of the subject can be used for monitoring. Additionally or alternatively, in a variation, client device usage parameters can be used for monitoring. Examples of client device usage parameters can include frequency of application switching, duration of time spent in association with each application login, screen time parameters, data usage associated with different applications and/or types of applications (e.g., social networking, creative, utility, travel, activity-related, etc.) executing on the client device of the subject, time of day of application usage, location of device usage, and other client device usage parameters.

Additionally or alternatively, in a variation, the system can process voice data and/or text communication data of the subject for monitoring and modifying interventions and program aspects. Examples of voice data can include voice sampling data from which emotional states can be extracted using voice processing models. In a related manner, natural language processing of textual data (e.g., from communication applications, from social networking applications) of the client device can be used to provide context for behaviors of the subject and/or assess emotional or cognitive states of the subject.

Additionally or alternatively, in a variation, electronic health record data can be used for monitoring. For instance, if the subject receives medical care, the online system can be configured to receive a notification providing information regarding the type of care the subject has received, and to use this data for monitoring statuses of the subject.

Additionally or alternatively, the system can include architecture for processing data from other sensors of the client device, devices in the environment of the subject, and/or wearable computing devices (described in Section 2 above) can be used for monitoring. Such device data can include activity data, location data, motion data, biometric data, and/or other data configured to provide context to behaviors associated with the health condition of the subject. In one example, motion data from motion of sensors of the client device can indicate that the user is sedentary, and may be experiencing symptoms that can be addressed with components of the intervention regimen. In another example, device usage data can indicate that the subject has been using a particular device (e.g., a tablet device in proximity to the subject, where use does not require extensive motion of the subject), in a fixed location (e.g., from GPS data), and in a prone position (e.g., from motion chip data), and may be experiencing GI health condition symptoms that can be addressed with components of the intervention regimen.

As such, active monitoring of patient states can be used to adjust delivery of intervention regimen modules in order appropriately meet the needs of the subject. Other data and combinations of data can, however, be used for monitoring.

### 3.4 Method—Positive Feedback Loops and Reinforcing Engagement

In relation to system components described above, an embodiment of the online system, in coordination with the network and a client device can, in response to at least one of the set of interactions and the health status progression, perform 250 an action configured to improve health and wellbeing of the subject with respect to the GI health condition. Block 250 functions to provide further customization of the intervention regimen, in order to improve personalization of delivered content to needs of the subject, in an adaptive manner. Block 250 can also function to increase engagement between the subject and the intervention regimen, in order to improve effectiveness of provided treatments and increase success of the subject in achieving his/her goals.

In embodiments, the action performed according to Block 250 can include one or more of: adjusting order of and/or content of intervention modules provided, where intervention types and content are described above; updating electronic health records (EHRs), personal health records (PHRs), and/or open medical records, for instance by writing to or modifying records whenever new information is generated regarding the user/subject/patient; providing and/or facilitating provision of supplemental interventions (e.g., hypnotherapy, physical exercises, medications, supplements, etc.) beyond standard content of the intervention regimen, for instance, under physician-guidance or treatment recommendations; generating and/or providing notifications to the subject regarding changes in behavior or health statuses; generating and/or providing notifications to entities (e.g., relatives, acquaintances having permission of the subject, health care providers, etc.) associated with the subject regarding changes in behavior or health statuses; and/or any other suitable action.

In embodiments, Block 250 can additionally or alternatively include functionality for increasing engagement of the subject with respect to interactions with content of the intervention regimen.

In variations, features for increasing engagement and optimal learning can include text-based functionality for self-monitoring and symptom tracking, where the system can process real time text interactions with provision of interactive tasks, which increases likelihood of patient responses. In more detail, specific descriptions self-reported by the subject can be used in subsequent portions of the intervention regimen to increase personalization of the intervention to drive engagement. Additionally or alternatively, features for increasing engagement and optimal learning can include features that mimic therapist/healthcare provider, or social group interactions (e.g., patient testimonials, clinician video content, etc.). Additionally or alternatively, features for increasing engagement and optimal learning can include features that link the subject's specific current problems (e.g., from Block 240) and/or challenges faced the subject as a trigger to notify the subject to interact with content of the intervention regimen and recommend appropriate skill for improving health states.

Additionally or alternatively, in variations, engagement can be promoted using one or more of: artificial reality tools (e.g., augmented reality platforms, virtual reality platforms) for reducing depression, anxiety, pain, and/or other symptoms; artificial intelligence-based coaching elements for driving interactions with the subject; smart assistants (e.g., Alexa™, Siri™, Google™ Assistant, etc.) for assisting the subject in relation to task management, gamification elements within intervention regimen-associated applications executing on the client device; gamification elements of other devices (e.g., smart toilet devices having interactive elements, such as buttons that control flushing and other subsystems, for promoting triggering of stool sample tracking in relation to various symptoms); smart pill devices and/or medication-dispensing devices that provide insights in an engaging manner in coordination with intervention regimen modules; adjustment of reinforcement schedules (e.g., in relation to reward sensitivity, positive reinforcement, negative reinforcement, etc.) for providing intervention regimen content to the subject; and other elements for increasing engagement.

As noted above, features for personalization and promoting engagement can be delivered within modules of the intervention regimen before and/or after monitoring of the subject according to Block 240.

### 3.5 Method—Additional Aspects, Reinforcement, and Predictive Models

As noted above, the method 200 can further include steps for detecting performance of activities associated with the intervention regimen, by the subject; reinforcing user performance or engagement with the intervention regimen; determining undesired levels of performance or engagement with the intervention regimen; and driving improved engagement with the intervention. For instance, in relation to various activities of the intervention regimen, the method 200 can include functionality for detecting performance or non-performance of activities (e.g., based on application engagement, based upon sensor-detected measures of activity, etc.). If the subject performs activities of the intervention regimen appropriately, the method 200 can include functionality for reinforcing performance through provision of various rewards (e.g., rests, rewards of monetary value, etc.). If the subject does not perform activities appropriately, the method 200 can include functionality for determining causes of non-performance (e.g., non-engaging content, external factors associated with the subject's life, etc.) and adjust content delivery, provide modified interventions, and/or adjust reinforcement schedules accordingly.

Furthermore, as indicated above, the method 200 can include functionality for developing and training predictive models for predicting states of the subject during the course of the intervention regimen, in order to improve chances of success in outcomes. The method 200 can thus include functionality for aggregation of training datasets from various data sources described above, and processing training datasets with one or more types of model architecture in order to improve predictions and/or selection of appropriate modules of the intervention regimen for delivery to the subject. Models associated with the method 200 can be defined within architecture of computing systems described above, and include elements for statistical analysis of data and/or machine learning.

In more detail related to model training, the method can include: generating a combined dataset upon applying a first set of transformations to an aggregate dataset including physiological data, behavioral data, environmental stress data, emotional data, and cognitive data from a set of users exhibiting a form of the GI condition 401 (e.g., where data is analogous to that described in relation to step 201 above); collecting a treatment dataset comprising treatment outcome labels (e.g., quantitative or qualitative labels describing efficacy of individual treatment components) associated with the subset of CBT components applied to the set of users 402; creating a first training dataset comprising the combined dataset and the treatment dataset 403; and training the model with the first training dataset 404. As such, the model can be structured and ultimately refined for receiving data objects associated with at least one of: physiological data, behavioral data, environmental stress data, emotional data, and cognitive data of the user, and returning a set of outputs comprising a selection of treatment subcomponents tagged with efficacy indicators.

Statistical analyses and/or machine learning algorithm(s) can be characterized by a learning style including any one or more of: supervised learning (e.g., using back propagation neural networks), unsupervised learning (e.g., K-means clustering), semi-supervised learning, reinforcement learning (e.g., using a Q-learning algorithm, using temporal difference learning, etc.), and any other suitable learning style.

Furthermore, any algorithm(s) can implement any one or more of: a regression algorithm, an instance-based method (e.g., k-nearest neighbor, learning vector quantization, self-organizing map, etc.), a regularization method, a decision tree learning method (e.g., classification and regression tree, chi-squared approach, random forest approach, multivariate adaptive approach, gradient boosting machine approach, etc.), a Bayesian method (e.g., nave Bayes, Bayesian belief network, etc.), a kernel method (e.g., a support vector machine, a linear discriminate analysis, etc.), a clustering method (e.g., k-means clustering), an associated rule learning algorithm (e.g., an Apriori algorithm), an artificial neural network model (e.g., a back-propagation method, a Hopfield network method, a learning vector quantization method, etc.), a deep learning algorithm (e.g., a Boltzmann machine, a convolution network method, a stacked auto-encoder method, etc.), a dimensionality reduction method (e.g., principal component analysis, partial least squares regression, etc.), an ensemble method (e.g., boosting, boot strapped aggregation, gradient boosting machine approach, etc.), and any suitable form of algorithm.

### 4. Conclusions

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A method comprising:
establishing an interface between a device and a user;
from the interface, receiving a set of signals associated with a gastrointestinal (GI) condition of the user, wherein the set of signals encodes physiological data, behavioral data, environmental stress data, emotional data, and cognitive data of the user;
determining a characterization of the GI condition upon processing the set of signals with a model;
based upon the characterization, modulating content of a treatment comprising a set of components, the set of components comprising a subset of cognitive behavioral therapy (CBT) components for improving a state of the user; and
administering the personalized treatment to the user.

2. The method of claim 1, wherein physiological data captured in the set of signals comprises pain characteristics, digestive characteristics, and defecation characteristics of the user tagged with symptom severity.

3. The method of claim 2, wherein behavioral data captured in the set of signals comprises social behavior characteristics of the user extracted from a communication subsystem of a mobile device of the user.

4. The method of claim 3, wherein cognitive data of the user captured in the set of signals comprises thought patterns associated with behaviors, anxiety characteristics, depression characteristics, and emotional characteristics of the user.

5. The method of claim 4, wherein determining the characterization comprises returning a mapping with a network of flows between a set of behaviors specific to the user, a set of thoughts specific to the user, a set of physiological symptoms specific to the user, a set of emotions specific to the user, and environmental triggers specific to the user, with returned outputs configured to disrupt flows of the network contributing to deterioration of symptoms of the user.

6. The method of claim 1, further comprising:
generating a combined dataset upon applying a first set of transformations to an aggregate dataset including physiological data, behavioral data, environmental stress data, emotional data, and cognitive data from a set of users exhibiting a form of the GI condition;
collecting a treatment dataset comprising treatment outcome labels associated with the subset of CBT components applied to the set of users;
creating a first training dataset comprising the combined dataset and the treatment dataset; and
training the model with the first training dataset.

7. The method of claim 6, wherein the model comprises architecture for receiving data objects associated with at least one of: physiological data, behavioral data, environmental stress data, emotional data, and cognitive data of the user, and returning a set of outputs comprising a selection of treatment subcomponents tagged with efficacy indicators, the method further comprising modulating content of the treatment based upon the selection returned by the model.

8. The method of claim 1, wherein the subset of CBT components comprises a prescription digital therapeutic (PDT), delivered through a client device of the user, with materials for adjusting gut and neurological activity of user having the GI condition, through pain management therapy, social behavior training, cognitive flexibility exercises, and behavioral repertoire changes.

9. The method of claim 8, wherein the treatment further comprises a subset of non-CBT components comprising at least one of: an antibiotic, an antidepressant, an antispasmodic, a 5-hydroxytryptamine 4 agonist, a laxative, an anti-diarrheal medication, a probiotic, and a selective C-2 chloride channel activator.

10. The method of claim 9, wherein modulating content of the treatment comprises adjusting an amount of the PDT relative to an amount of the subset of non-CBT components provided to the user, based upon a returned output of the model.

11. The method of claim 1, wherein providing the treatment comprises generating instructions for adjusting activation of an environmental control device in an environment of the user, in coordination with monitoring a change in pain symptoms of the user, thereby producing an adjustment in pain intensity and duration associated with the GI condition of the user.

12. The method of claim 11, wherein the environmental control device comprises operation modes for at least one of: an audio output, a thermal parameter adjustment, a visually-observed output, a haptic output, and a light output in the environment of the user.

13. The method of claim 1, wherein providing the treatment comprises generating a scripted communication for transmission to an entity associated with the user, in coordination with monitoring a change in a physiological symptom of the user, thereby preventing an adverse social interaction involving the user, due to the GI condition.

14. The method of claim 13, further comprising receiving, through the interface, a reward sensitivity dataset characterizing motivation and reinforcement contingencies and behavior of the user, and modulating aspects of the treatment upon processing the reward sensitivity dataset with the model.

15. A system comprising:
an input device providing an interface with a user; and
a processing subsystem in communication with the input device and comprising a non-transitory computer-readable medium comprising instructions stored thereon, that when executed by the processing subsystem perform one or more steps of:
receiving from the interface, a set of signals associated with a gastrointestinal (GI) condition of the user, wherein the set of signals encodes physiological data, behavioral data, environmental stress data, emotional data, and cognitive data of the user;
determining a characterization of the GI condition upon processing the set of signals with a model;
based upon the characterization, modulating content of a treatment comprising a set of components, the set of components comprising a subset of cognitive behavioral therapy (CBT) components for improving a state of the user; and
administering the treatment to the user.

16. The system of claim 15, further comprising an output device comprising an operation mode for administering the treatment to the user.

17. The system of claim 16, wherein at least one of the output device and the treatment is provided to the user as a prescription therapeutic available upon diagnosis of the GI condition of the user.

18. The system of claim 17, wherein the output device comprises an environmental control device in an environment of the user, wherein the processing subsystem further comprises instructions for adjusting the operation mode in coordination with monitoring a change in pain symptoms of the user, thereby producing an adjustment in pain intensity and duration associated with the GI condition of the user, and wherein the operation mode provides at least one of: an audio output, a thermal parameter adjustment, a visually-observed output, a haptic output, and a light output in the environment.

19. The system of claim 17, wherein the output device comprises a communication device comprising architecture for transmitting communications between the user and an entity associated with the user, wherein the processing subsystem further comprises instructions for generating a scripted communication for transmission to an entity associated with the user, in coordination with monitoring a change in a physiological symptom of the user.

20. The system of claim 15, wherein the input device comprises a set of biometric sensors in communication with the user and configured to generate physiological data, from the user with the GI condition.
